(19) United States
(12) Patent Application Publication
(10) Pub. No.: US 2023/0215525 A1
(43) Pub. Date: Jul. 6, 2023

(54) HEALTH INFORMATION EXCHANGE AND INTERACTION PLATFORM

(71) Applicant: MFIC Ventures, Jasper, GA (US)

(72) Inventor: Matthew Standish, Canton, GA (US)

(21) Appl. No.: 18/054,888

(22) Filed: Nov. 11, 2022

Related U.S. Application Data
(60) Provisional application No. 63/278,895, filed on Nov. 12, 2021.

(51) Int. Cl. G16H 10/60

(52) U.S. Cl. CPC G16H 10/60; G06Q 30/0276

(57) ABSTRACT

In various aspects, the present disclosure provides a computer-implemented method for targeted patient interactions. In one aspect, the method is configured to receive, by a processor, a patient identifier, wherein the patient identifier contains identifying data for a new patient. The method collects, by at least one channel, patient data associated with the new patient, wherein the patient data is collected from at least one partner service. The method analyzes, by an analytical engine, the patient data. The method generates, by the processor, at least one targeted patient event, wherein the targeted patient event is generated by the analysis of the patient data.

### CROSS-REFERENCE TO RELATED APPLICATION

This applications claims priority to U.S. Provisional Patent Application No. 63/278,895, filed Nov. 12, 2021, and entitled “Health Information Exchange and Interaction Platform,” the disclosure of which is incorporated herein by reference in its entirety.

### BACKGROUND

There are many challenges with health care and healthcare providers that include privacy, delivery of care, telemedicine, acquisition and aggregation of data using devices and apps, reliability of medical information, and more. These challenges to health care and the insurance industry are omnipresent and the effects on privacy and the understanding of our health data are not understood using today's healthcare platforms. Today, health services use incomplete sources of data to promote need-based medical services. Real-time health data today integrated with historic electronic health records does not exist. For example, health data and analytics from diet/calorie-counting, wearables, and fitness apps “trap” critical wellness data inside devices and behind subscription walls. Unable to communicate this information to a single platform, such data is often flawed leading to inaccurate false positives and misleading and potentially harmful outcomes. It is also increasingly difficult to trust primary care doctors as they often lack testing and results derived from data and science and rely on marketing by pharmaceutical companies or politically biased government agencies.

TeleHealth (referred to herein as “Telemedicine”) has become increasingly prevalent, especially, during the pandemic that began in 2020. Telemedicine generally refers to the use of virtual, computer-based, and mobile technologies and sensors to turn communication into an interactive dialogue between Provider and Patient data. While primary care medicine is progressing towards virtual-based medicine, current approaches are not based on a deep understanding of the use and interrelationships of the underlying telecommunications technologies (e.g., 5G+, WiFi 6+, Bluetooth Low Energy (BLE)) and devices, e.g., wearables, Internet of Things (IoT), and other sensors that allow for the specific low draw of power from nano-devices that attach to a person, skin, clothing or as a wearable or micro-medical device(s). Data from these devices and their respective communication pathways are not integrated in a way to properly support virtual diagnoses, prescriptions, diet et al. because the available real-time and historical data and the associated analytics are not used. For example, a virtual medicine consult may use data and analytics derived from smart scale(s), Body Mass Index (BMI), Galvanic skin response (GSR), Blood Pressure data, DNA analysis, recent blood labs, and other real-time analytics such as wearable data (e.g., Steps, Intensity Activity from heartbeat information, stress level), however, this information is not available. In particular, telemedicine lacks a set of Internet or computer-based applications that provide real-time information about the patient both before a virtual patient visit is initiated that exchanges Electronic Health Records (EHRs), prescription and vaccine records, real-time and historical sensor data, to build a patient's health Persona (described below) which is based on analytics of unstructured data sets or past and previous real-time health information calculating potential outcomes for the Provider to analyze before, during and after a Telemedicine session is initiated. The failure to use wearables, sensors, and smart IoT device(s) that provide a picture of a patient's health or baseline state as a context of diagnosis is completely lost by conventional Telemedicine. Thus, not having access to key unstructured, real-time patient information is a drawback within Telemedicine.

Conventional patient portals, customer relationship management (CRM) systems, CRM Plugins, Social CRM, Business Intelligence, or Master Data Management sectors have limitations, such as old designs that fail to address security settings, firewall settings, and encryption services. Never-mind hard-coded and frail CRM systems that fail to take a real-time mobile-based approach and fail to focus on analytics, patient context, internal processes, reporting, and procedures. Conventional CRM systems lack a real-time Al and the ability to use unstructured data sources. For example, these systems use data, such as business Intelligence sources and historical data that is indirect and not actionable. Yet further, conventional systems do not provide for data management that creates a single view of the customer. They are not able to consider the timeliness of patient appointments, which is a consideration in providing adequate healthcare. As a result, big data is getting exponentially bigger while continuing to under-deliver on the promise of being actionable and generating incremental revenue. Thus, there is a need for enterprise-grade CRM solutions that offer simplicity, automation, focused on being a historical repository and with real-time Patient Al is a bridge to new revenue and new mobile TeleHealth markets. The health exchange platform of the present disclosure overcomes the above limitations and addresses needs in the art.

### SUMMARY

The present disclosure is directed to a health exchange platform that provides an infrastructure for connecting predictive health results using analytics derived from disparate data sources (e.g., wearables, patent health records, etc.) to healthcare providers, health systems pharmacies, and/or insurance companies with a patient's explicit opt-in. In particular, the health exchange platform deploys “Health Behaviors” by combining IoT sensor(s), EHRs, and analytics with Artificial Intelligence (AI) based on exercise and DNA. The health exchange platform further combines real-time health patterns with genetic pharmacological data identifying medicine interactions and integrates health care and commercial sensors through a mobile application. A health exchange platform is a mobile phone based unique software as a service (SaaS) Platform that integrates and standardizes wearable and sensor analytics using machine learning (ML).

The present disclosure further provides methods and systems for predictive analytics opportunities by identifying the patient/customer and where they are in the patient/customer lifecycle (i.e., new, old, VIP); capturing real-time statistics on user interactions with the brand, category, and specific product or services; statistically correlating the history of the brand, product, and service mentions and interactions; and engaging patient/customers with offers and promotions relative to real-time purchase decisions, commentary, and influencer rating.

The health exchange platform may explicitly identify patient needs, determine the patient's intent, develops the patient's propensity of care or Health Outcomes (HO), determine if the patient is in the market today, and interact with the patient across multiple channels of communication. Thus, a patient's concerns and needs are managed at many different places in the value chain at scale. In pursuit of creating a more economical way to conduct consumer interaction, the health exchange platform provides an outcome-based approach to an online medical needs platform focused on the identification of consumer market patient needs to be followed by automatic recommendations and fulfillment for revenue conversion. The health exchange platform is designed to effectively and efficiently patient needs for their health services, insurance plans, and services. As will be further described below, the health exchange platform also provides for identity and access management; role and responsibility privacy-based sign-in; real-time 360° contextual understanding, patient needs signal algorithms; identification of intent and propensity of care, and patient recommendation and automation; segmentation and delivery. The health exchange platform also provides a modern store of health information that addresses patient concerns.

The health exchange platform of the present disclosure also addresses the need for a solution that addresses the need to aggregate expanding amounts of consumer data from social, mobile, and CRM systems, and leverage that data for the benefit of the enterprise and patient outcomes. The health exchange platform also reduces the ability of healthcare data to be sold or misused without consent. The health exchange platform of the present disclosure makes it easier for patients to visualize and securely store patient health records with encryption at their direction. A patient can aggregate health records (e.g., EHRs) from multiple providers alongside personally generated data creating a more holistic predictive view of health outcomes out of the predatory view of aggressive advertisers and spam lists that fail to comply with HIPPA+.

Addressing the above-noted limitations of conventional systems, the health exchange platform may ingest consumer usage data from social media and mobile applications, which is a valuable source for real-time information about consumer behavior, consideration, and purchasing power. This information, used in conjunction with existing CRM data, can fuel a timelier and more effective “patient needs” based medical needs approach (versus “supply”) by enhancing consumer patient personas leading to improved targeting and revenue conversion. The enterprise business problem is how to consolidate, analyze and leverage all this data—and the contextual information it contains—in an efficient and actionable way.

Thus, in view of the above and other aspects of the disclosure, a computer-implemented method for targeted patient interactions is disclosed. The method includes receiving, by a processor, a patient identifier, wherein the patient identifier contains identifying data for a new patient; collecting, by at least one channel, patient data associated with the new patient, wherein the patient data is collected from at least one partner service; analyzing, by an analytical engine, the patient data; and generating, by the processor, at least one targeted patient event, wherein the targeted patient event is generated by the analysis of the patient data.

In accordance with other aspects, a health exchange platform system is disclosed that includes a cloud infrastructure layer that includes storage capabilities and networking capabilities and a data management layer that includes an Application Programming Interface (API) and communication layer, a business process management layer, a rules layer, an ID management layer, a data access layer, a Lightweight Directory Access Protocol (LDAP) layer, and a database federation layer. A patient identifier uses Artificial Intelligence and real-time IoT data for a new patient is received by the cloud infrastructure layer and patient data associated with the new patient and at least one channel and at least one partner service is collected by the data management layer. An analytical engine in the data management layer analyzes the patient data to generate at least one targeted patient event from the patient data.

This summary is provided to introduce a selection of concepts in a simplified form that is further described below in the detailed description. This summary is not intended to identify key features or essential features of the claimed subject matter, nor intended to limit the scope of the claimed subject matter.

### BRIEF DESCRIPTION OF THE DRAWINGS

The foregoing summary, as well as the following detailed description of illustrative implementations, is better understood when read in conjunction with the appended drawings. For the purpose of illustrating the implementations, there is shown in the drawings example constructions of the implementations; however, the implementations are not limited to the specific methods and instrumentalities disclosed. In the drawings:

FIG. 1 illustrates an example implementation of a Health Logical Architecture to implement a health exchange platform;

FIG. 2 shows an example implementation of the Ingestion Engine to create a patient person using normative discourse measures;

FIG. 3 shows an example implementation of a health interaction reference data model;

FIG. 4 shows an example implementation of a health interaction reference data model with identity management;

FIG. 5 shows an example implementation of a patient lifecycle of a patient with actionable analytics and an intent engine with event-based outcomes;

FIG. 6 shows an example implementation of a future state SaaS Platform & Approach;

FIG. 7 shows an example implementation of a general computing network on which the health exchanges and methods may be implemented;

FIG. 8 shows an example implementation of a patient device(s) interfacing with Exchanges and services that can integrate with the health exchange;

FIG. 9 illustrates an example interaction diagram and database attributes to define patient(s);

FIGS. 10-21 illustrate example user interfaces associated with an end-user application to interact with the health exchange platform of FIG. 1; and

FIG. 22 illustrates an example computing environment in which the present disclosure may be implemented.

### DETAILED DESCRIPTION

It is to be understood that this disclosure is not limited to particular aspects or implementations described, and as such may vary. It is also to be understood that the terminology used herein is for the purpose of describing particular aspects or implementations only, and is not intended to be limiting, since the scope of the method and Exchange for phonic learning using mobile and sensor computing device(s) are defined only by the appended claims. A general overview of the various implementations is provided in the description immediately following, and implementations of the various implementations are provided with reference to the figures. The overall scope of the present disclosure is provided in the appended claims.

Platform Overview— Patient-User Perspective

The present disclosure is directed to a health exchange platform that provides an infrastructure for connecting predictive health results using analytics derived from disparate data sources (e.g., wearables, patent health records, etc.) to healthcare providers, health systems pharmacies, and/or insurance companies with a patient's explicit opt-in. In particular, the health exchange platform deploys “Health Behaviors” by combining IoT sensor(s), EHRs, and analytics with Artificial Intelligence (AI) based on exercise and DNA. The health exchange platform further combines real-time health patterns with genetic pharmacological data identifying medicine interactions and integrates health care and commercial sensors through a mobile application. A health exchange platform is a unique software as a service (SaaS) Platform that integrates and standardizes wearable and sensor analytics using machine learning (ML).

The health exchange platform may explicitly identify patient needs, determine the patient's intent, develops the patient's propensity of care or Health Outcomes (HO), determine if the patient is in the market today, and interact with the patient across multiple channels of communication. Thus, a patient's concerns and needs are managed at many different places in the value chain at scale. In pursuit of creating a more economical way to conduct consumer interaction, the health exchange platform provides an outcome-based approach to an online medical needs platform focused on the identification of consumer market patient needs to be followed by automatic recommendations and fulfillment for revenue conversion. The health exchange platform is designed to effectively and efficiently patient needs for their health services, insurance plans, and services. As will be further described below, the health exchange platform also provides for identity and access management; role and responsibility privacy-based sign-in; real-time 360° contextual understanding, patient needs signal algorithms; identification of intent and propensity of care, and patient recommendation and automation; segmentation and delivery. The health exchange platform also provides a modern store of health information that addresses patient concerns.

The health exchange platform of the present disclosure also addresses the need for a solution that addresses the need to aggregate expanding amounts of consumer data from social, mobile, and CRM systems, and leverage that data for the benefit of the enterprise and patient outcomes. The health exchange platform also reduces the ability of healthcare data to be sold or misused without consent. The health exchange platform of the present disclosure makes it easier for patients to visualize and securely store patient health records with encryption at their direction. A patient can aggregate health records (EHRs) from multiple providers alongside personally generated data creating a more holistic predictive view of health outcomes out of the predatory view of aggressive advertisers and spam lists that fail to comply with HIPPA+.

Addressing the above-noted limitations of conventional systems, the health exchange platform may ingest consumer usage data from social media and mobile applications, which is a valuable source for real-time information about consumer behavior, consideration, and purchasing power. This information, used in conjunction with existing CRM data, can fuel a timelier and more effective “patient needs” based medical needs approach (versus “supply”) by enhancing consumer patient personas leading to improved targeting and revenue conversion. The enterprise business problem is how to consolidate, analyze and leverage all this data—and the contextual information it contains—in an efficient and actionable way.

Platform Overview— Provider-Operator Perspective

In an aspect, the health exchange platform is a patient needs identification platform that enables enterprise digital marketers to identify and convert real-time consumer patient needs into revenue while respecting consumer privacy. The health exchange platform simplifies the identification and understanding of consumer “patient needs signals” collected from multiple data sources, including unstructured and largely unused data from social media and mobile applications as well as structured data from CRM systems. The patient needs signals are analyzed to identify highly qualified prospects with real-time interest and intent for specific health services, insurance plans, and services. For operators of the health exchange platform, the platform packages this information in robust consumer patient personas called “Vitality Plus” that represent individuals, groups, events, and communities. Vitality Plus data is continually updated so marketers can reliably identify and interact with qualified prospects based on their propensity to buy and revenue potential. The health exchange platform provides for visualization of patient needs curves while segmenting and recommending qualified prospects for automatic delivery of offers and information using social media direct messages, wall posts and tweets, or existing email systems. Client platform utilization and results are measured, tracked, and reported to assess effectiveness and return on effectiveness.

Being a software-as-a-service (SaaS) platform, the health exchange platform is a high velocity—low inertia offering requiring little to no IT department involvement. Medical needs teams can quickly deploy “Exchange” (a patient needs identification) and begin using actionable analytics in a matter of days without the need for custom data models, professional services, or extensive IT support. The health exchange platform may be envisioned as having the following, non-limiting, functionalities.

Channel Scope—includes the universe of unstructured data sources that can supplement traditional structured data and mobile health and exercise application sources.

Health Advocate—Patients and their telemedicine physicians will have access to our Health Advocate App which is the Single Source of Truth for all historical and real-time data/health events/sensors and your virtual advocate to improve health outcomes with your physician/specialist.

Predictive Intelligence—the health exchange platform's artificial intelligence (AI) platform uses real-time integration with sensors to supply predictive outcomes based on health records (EHRs), historical health data, sensor intelligence, DNA testing, blood tests, vaccinations, past surgeries, and current health conditions.

Single Source of Truth—provides “health outcomes,” and sets personal data free that is otherwise siloed in apps and devices to create a platform for patent data security and knowledge

Multi-channel data and deep consumer insights—ingests unstructured data from mobile applications and social media channels and adds structured data from existing CRM systems (event attendance, addresses, etc.) to create single, comprehensive, contextual views of consumers called “Vitality Plus” for individuals, groups, events, and communities.

Prediction of consumer intent—the health exchange platform uses rich consumer profiles and context to enable the prediction of consumer intent, which can be proactively addressed with highly targeted and relevant, automated medical needs activity using the platform.

Real-time opportunity identification and qualification—the health exchange platform aggregates and assess consumer conversations across multiple channels to identify consumer patient needs signals indicating real-time intent and propensity to buy. Quantifies the monetary value of consumer propensity to buy for enhanced targeting.

Automatic interaction and revenue conversion—the health exchange platform maintains a portfolio of offers and criteria for targeting and delivery. Automatically aligns and executes offer delivery to real-time prospects.

Easy to use and fully integrated platform—the health exchange platform provides single access to platform capabilities through a SaaS login. The health exchange platform's intuitive interface leverages visual business intelligence to enable operators to visualize the patient needs curves of their business while segmenting and recommending prospects for offer delivery.

Fast deployment and competitive pricing—the health exchange platform deploys with little to no IT organization involvement. Client accounts are on-boarded, and patients are trained in as little as a few days.

In medicine today, inaccessible data is not the only problem. Different departments, Providers, Health Care Exchanges, Ambulatory Exchanges, Hospitals, Pharmacies and other healthcare and pharmaceutical channels find themselves with contradictory views of a patient based upon contradicting, old or bad data. The health exchange platform provides for outcome-based medicine using Artificial Intelligence and Predictive Analytics provide an emerging channel of virtual medicine, virtual patient care, real-time (un)structured analytics, Al and greater collaboration between Providers, Patients, Healthcare Systems and Insurance Companies. Further, the health exchange platform provides a single source of truth about the Patient called a “Persona” attached to a health “Exchange” that embraces the real-time nature of diagnostic patient attributes as defined above and provides a more cost-effective, more flexible, more accessible, and compelling patient experience and/or treatment plan.

Consolidate View of Patient Data—Persona Plus

In accordance with aspects of the disclosure, the health exchange platform provides real-time multi-source data federation that integrates static patient CRM data with dynamic Telemedicine, EHR, sensor, unstructured, structured, and mobile application data. The solution uses cutting-edge data management techniques to gather raw or unstructured data about patients from multiple channels, then segments that data into patient personas called “Persona Plus” based on the identified patient needs, outcomes, or “Health Signals.” The identified patient needs signals from patterns of intent that emerge through an analysis of the online behavior of individuals, groups, and communities.

Persona Plus are federated datasets that present a consolidated single view of a patient's real-time behavior and patient needs for health services, insurance plans, research, generational trends/information and services across multiple data sources. The health exchange platform analyzes various characteristics of generated Personas and machine-learning predictive analytics to build the patient personas according to their propensity of care (PoC). This PoC is to grade the Persona for pending or upcoming issues related to their baseline Persona. In tandem coverage, genetic issues, past surgeries or treatments and coverage are evaluated. Conversely, potential patient revenue or margin is also calculated for a provider or system that informs local treatment providers of potential patients and Insurance companies of a patient's needs both real-time and projected.

The health exchange platform's Patient Persona is critically powerful in the ability to diagnose based on contextual information. Such information can be categorized by age, generation, health issue, health coverage status, current or future or conflicting medication types, or by handicap, disease et al. For example, the health exchange platform can be adapted to realize an individual's generational potential trending issues, diagnosis, and remedies. An end user would not have to expend time on Internet searches, but rather could have curated content focused on you contextually reflective of your Patient Persona through the health exchange platform. The health exchange platform does not create the information but rather it ranks and pulls information from legitimate sources using an algorithm that ranks and stacks credibility of all medical issues you can opt into.

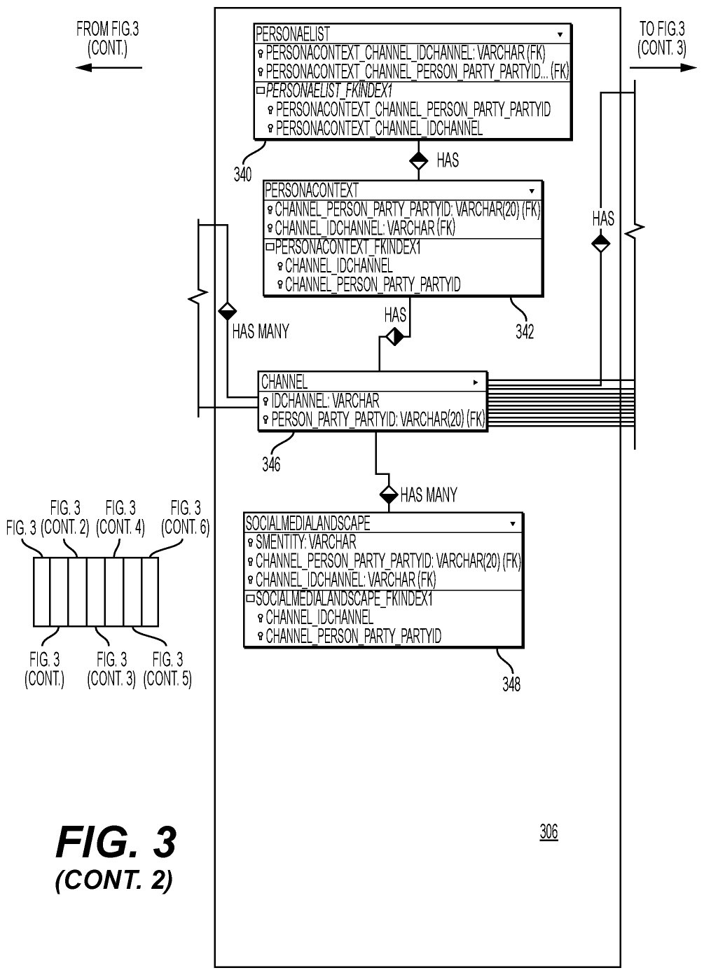

In an implementation of the present disclosure, health analytics is provided that is based on a very deep understanding of patients that includes location, structured data, unstructured data, and outcome based derivative data and the ability to quantify such measurements into a Patient Persona through ID Reconciliation connected to the health exchange platform which needs to be able to support any application that requires Identity Reconciliation to build a “Persona” about a patient—for both known and unknown continuously attempting to resolve unknown IDs to known. This is accomplished by providing a means to capture and execute business rules to support the movement of data monitoring and managing Exchanges and jobs during execution with a variety of processing engines. In various aspects, the present disclosure is directed to telecommunications, retail health, patient product(s), pharmaceuticals, prescriptions, financial/insurance, and healthcare patients, amongst others. In the example of telecommunications or TeleHealth patients, the objective(s) are high-touch business outcome-based patient models to create patient intimacy, understanding, and outcomes while automating health and patient interactions as in FIG. 5. In one aspect, the present disclosure is directed to managing the analytics of unstructured data in the form of monetizing or reducing costs of the unstructured data with additional context that creates a Health Persona that supports the use cases while providing depth and dimension.

From an operator perspective, the health exchange platform may offer a “white-label” model where an insurer, provider, hospital or ambulatory/health system, or specialists such as health coaches, and dieticians can leverage the health exchange platform as a “SaaS” or Software as a Service and use an encrypted database and encrypted data within the database using their own branding, labeling, copyrights and trademarks to leverage the system based on security system called (IDAM) Identity and Access Management wherein role(s) are assigned to the Patients based on the following:

1. Use Case

2. Security level

3. Trust level

4. Coach/Life Coach/Dietician

5 Family Member(s) or Legal Guarding

6. Administrator for the SaaS client

7. The administrator of the platform

Families may use the platform across geography or time zones to remain aware of any health events for those loved ones with heart conditions, diabetes, bariatric surgery or those who recently have been released from the hospital. Further, a consumer can use the health exchange platform for identifying lifestyle, exercise, and diet trends and how that integrates with their personal physician, trainer or health coach. This allows for a single license for individuals to download an associated app for use on a mobile device that evolves with market trends and social media. This overcomes limitations created by the fragmented market of sensors trapped in separate apps that do not work very well or integrated for the Bluetooth, hospital, pharmacy, or (BLE) grade of sensors. Now individuals will have an integrated view of doctor's appointments, dietician's appointments, hormonal appointments, etc. that are all linked to all DNA statistics from services like “23 and Me” or blood tests or stool tests et cetera from third parties lab providers.

The health exchange platform provides marketing entities such services or algorithms predict the intent and action(s) of allowing marketers to understand their patients' patient needs while automating actionable next steps for engaging patients, such as offers, informational emails, and Telemedicine messages. The patient needs identification platform enables digital marketers and sales teams to proactively identify and convert market patient needs into revenue.

The health exchange platform provides a single source of ranked credibility to sift through biases created by governmental funding in academics, healthcare, and pharmaceuticals, etc., to identify the influences of medical and health-based information based on source, headline, content using sentiment analysis and deep research of sources.

The health exchange platform “Persona” and the health exchange platform “Exchange” bring together a patient's EHRs, health insurance, financial state, scripts, geographically available care coverage, and patient's prescribed medical products; i.e., Oxygen, pharmaceutical sensors, medicine, vaccines, DNA with the ability to quantify measurements on a real-time basis Providers' understanding of large amounts of unstructured or dark data sets described above and herein. This introduces generational event-based medicine, which normalizes and simplifies the process of finding and evaluating care options and timeliness of appointments.

The health exchange platform may ingest the following non-limiting types of data from the following non-limiting list of sources:

Uploads

DNA— A123 | Ancestry | Trait Services

LAB TESTS— Everlywell | Silverberry Genomix | Cologuard

Smart Scales—Korescale | QARDIO | Withings | Garmin | iHealth | RedOver (Amazon

Top Scale)

API Integrations

Wearables—Fitbit | GoogleFit | Garmin | Withings | Apple | Beurer | Samsung

Sensors—Temperature Sensors | Gas | Pressure Sensors | Accelerometers | Proximity

Sensors | GPS | Gyroscope | Optical | Infrared | IoT Sensors | OVELDA Human Presence

Sensor | Qeexo AutoML (for Cortex-MO and MO+) | XENSIV™ 60 GHz radar sensor

Medical

OmniVision Technologies—OHOTA OVMed® Medical Image Sensor

TEKTELIC eDoctor—TEKTELIC Communications Inc.

Standard for IoT

MIL-STD 202G Method 105C Barometric (9/12/63) describes test procedures for barometric sensors used in high-altitude aircraft.

Standards of the International Standards Organization (ISO) that fall under ISO/TC 30/SC 2 differential device(s), ISO 21750:2006, and road vehicles—safety enhancement in conjunction with tire inflation monitoring.

ISO 15500-2:2012 (en) road vehicles—compressed natural gas (CNG) fuel Exchange components, which have two parts that specifically involve sensing which is: performance and general test methods and part 8 indicators.

A certification program by NSF International specifying the safety and quality requirements of automobiles for wheel tire monitoring sensors for the aftermarket parts industry.

Standard specification for transducers, differential, electrical and fiber-optic, and active standard, ASTM F2070 issued by the American Society for Testing and Materials (ASTM) International that covers the requirements of differential transducers for general applications.

In another implementation, the health exchange platform includes the ability to distribute and share website content with social networks. In accordance with the current approach, the health exchange platform targets at least four business scenarios: event analytics and outcome engine consists of: a repository; means for the enrichment of events; business overlay with conformed dimension; rules engines that allow automation based on data ingestion, ID-reconciliation, health privacy persona.

In yet another implementation, the health exchange platform includes a “Patient Complaint and Support Management” solution that enables Enterprises to use Telemedicine to provide the outstanding service levels and immediate, a personal response that patients increasingly expect. This packaged software lets a company capture comments from patients, no matter what the source. The Exchange identifies the patient and the nature of the complaint or request using Al and semantic technology. The video call or secure comment's content is evaluated and sent to an appropriate Doctor or Specialist, who responds to the patient via the same media. Once the problem is resolved, the patient's EHR record is automatically updated.

In another implementation, the health exchange platform includes a “Brand Management” package that helps Enterprises listen to the voice of the patient. The Exchange searches Telemedicine for comments, analyzes unstructured data, and reports on its discoveries concerning patient sentiments, issues, and trends. Of primary importance is a mechanism for identifying instances that could damage the brand. Type, key influencers, and key medical sites like “clevelandclinic.com” or “WedMD.com” categorize conversations. In response, the company can respond and actively engage patients in a dialogue about its products and services. Issues are resolved, myths are debunked, and relevant information is shared. This intimate response encourages patient satisfaction and loyalty

In another implementation, the health exchange platform includes an “Insurance Rebate, Voucher, and Point Administration” package that enables companies to use Telemedicine for healthcare campaigns and promotions, and then confirm their ability to address patients' expectations. Personalization and knowledge of location allow for the delivery of services that more closely matches the patient's interests. For example, communications can be initiated over the patient's preferred channel. Cross-selling and up-selling recommendations can be based on a patient's interests. And the information gathered can help application developers or the operator to build more intimate relationships with the patient.

In another implementation, the health exchange platform includes a “Sales, Upgrades, and Offers Automation” package that helps organizations reduce costs and increase revenue by enabling the automation of business processes, flow, and the implementation of sales force automation. The strategy allows the healthcare organization to manage, automate, and optimize campaigns across patient segments; types of campaigns, and healthcare touch points. Store operations can improve the in-store experience based on what patients are saying about the shopping experience, employees, return policy, store layout, and more. The Chief Executive Officers (CXOs) can learn how shoppers feel about products, pricing, and quality while promoting brand health and community relations. In the process, patients become loyal product “experts” and generate reusable content for healthcare purposes.

In another implementation, the health exchange platform includes a Social Interaction Platform that can integrate existing IT Exchanges with Web click-entry and exit points for campaigns, vouchers, health vouchers, and special discounts. Using our unstructured Health Persona and Interaction Engine we can Group by Events, Time of Year, or any customized Segment Analysis.

In another implementation, the health exchange platform processes the linguistics of tweets for specific keywords such as phrases, company, or product names. The Intent Engine may measure and can set alerts to notify employees or stakeholders of significant changes in tweet activity-grouping authors by brand status to identify brand detractors and advocates. The Intent Engine may combine EHR and Health Persona data into one location, where patients can sort, analyze and act upon it within a shared service library.

In an aspect, the health exchange platform implements shared services which is a library of “common code”—so Shared Services would house the JPA data model [com.hunuuhealth.datamodels] and the Data Service [com.hunuuhealth.dataservices] classes in the Shared Services project. A developer creates specialized utilities inside of the Shared Services layer such that specialized utility classes could be message processing algorithms, message processing filters, message processing rules, etc.

As such, the health exchange platform implements algorithms and processes as “plugin components” that live in the core library can be executed, consumed, extended, or implemented by any other tool, web service, or process that accesses the shared service library. Libraries should be in proper java “packages” for example: “com.hunuuhealth.messagefilters” and the library coded as part of the shared services library. The “Technology-Stack-Overview” is the data tier as described in the shared serviced library using JPA Entity classes and developer codes a data service class to access the entity. Next, a separate independent project is coded that is compiled as a library [JAR file]. The jar file imports shared services for database access and are the classes mechanism(s) needed to implement Health Analytics. Health Analytics is a jar file that accesses its own data via the aforementioned-shared services. Health Analytics can be imported into any other tool or used just like it is a “Shared Component.” This way, the health exchange platform creates libraries of reusable and extensible code bases and the libraries use each other to satisfy dependencies. Once the processing library is created, its goal is to consume and leverage said library and method(s) at the point and/or place needed inside one of the “Deployed Tooling” projects; i.e.: import com.hunuuhealth.analytics.analyzeMessageTrends;

### Example Environment

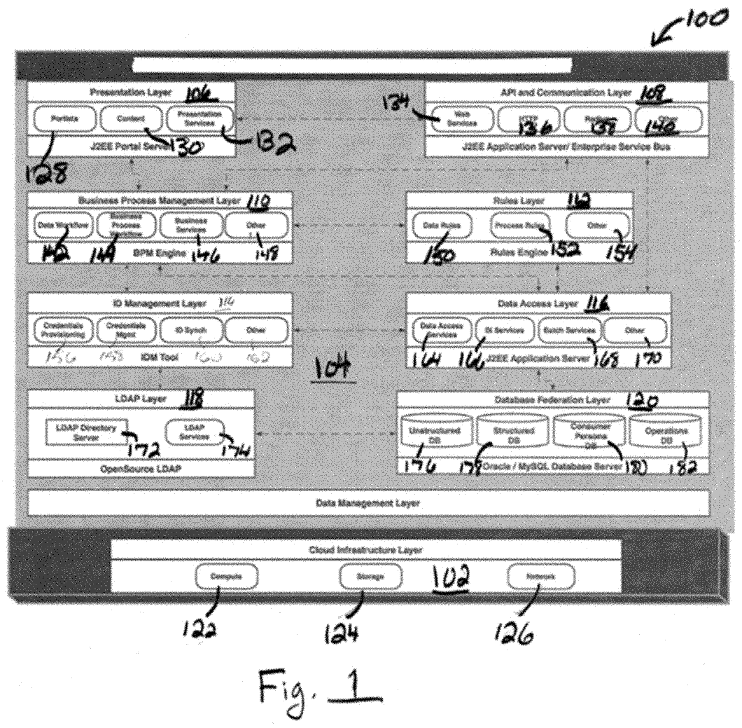

With reference to FIG. 1, there is illustrated in FIG. 1 an implementation of a health exchange platform logical architecture 100. As shown in FIG. 1, the health exchange platform logical architecture 100 may include a cloud infrastructure layer 102 and a data management layer 104. The cloud infrastructure layer 102 may include computing capabilities 122, storage capabilities 124, and networking capabilities 126. The cloud infrastructure layer 102 may be in data communication with the data management layer 104.

In some implementations, the data management layer 104 may include several sub exchanges, or layers, including, but not limited to a presentation layer 106, an Application Programming Interface (API) and communication layer 108, a business process management layer 110, a rules layer 112, an ID management layer 114, a data access layer 116, a Lightweight Directory Access Protocol (LDAP) layer, and a database federation layer 120. The layers that include the data management layer 104 may be in data communication with one or more other layers contained within the data management layer 104.

In some implementations, the data management layer 104 includes a presentation layer 106. The presentation layer 106 may include portlets 128, content 130, or presentation services 132. A portlet 128 is a pluggable patient interface software component that is managed or displayed in a web portal. The portlet 128 produces fragments of markup code that are aggregated into a portal. Typically, following the desktop metaphor, a portal page is displayed as a collection of non-overlapping portlet windows, where each of the portlet 128 window displays a portlet. Hence, the portlet 128 (or collection of portlets) resembles a web-based application that is hosted in a portal. Some examples of portlet applications include but are not limited to; email, health reports, health discussion forums, and prescription news.

In some implementations, the presentation layer 106 is implemented as a Java Platform Enterprise Edition (J2EE) Portal Server. Those skilled in the art will recognize that the present disclosure is not so limited and the presentation layer 106 may be implemented in any suitable programming language that allows for the management of portlets 128, content 130, and presentation services 132. Examples of other programming languages that may be used to implement the presentation layer 106 include, but are not limited to, Hypertext Preprocessor (PHP), Apache, or Java programming languages.

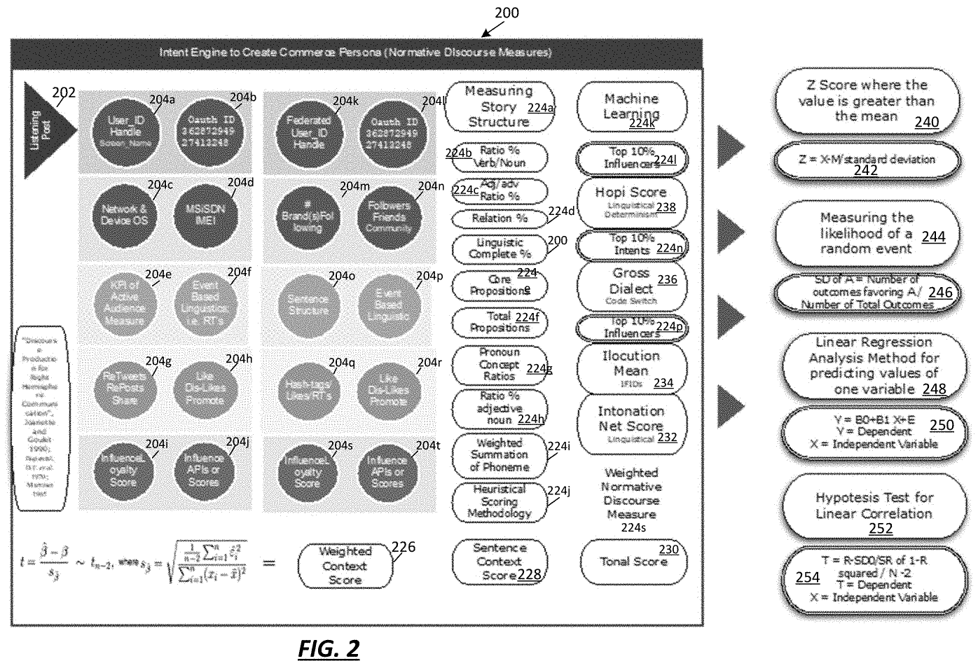

In accordance with aspects of the disclosure, the health exchange platform may collect categories of customer data as described in FIGS. 2, 3, and 5 below. For example, four types may be contemplated; however, additional or fewer types may be collected. The first type of data is associated with identity. Herein, an identity is mapped to a physical (or corporate) individual and is determined by the collection of facts about that individual that distinguishes them sufficiently from other individuals. This means that the health exchange platform's current view of an identity may be based on an individual's name, social security number, current address, credit score, etc. If that individual becomes a subscriber, then the various pieces of data are collected, by employees using computer systems, and becomes an individual's account. The other types of data associated with the individual may include the details of the offering used by the subscriber and the context in which that offering is used. The product of such usage and context data associated with an individual is a set of behavioral information that grows each time an offering is used.

An account record captures the identity and offering information that fundamentally reflects the terms of a contract between the customer and the health exchange platform, e.g., the length of the contract; the services that can be consumed by the customer; a record of how much the customer will pay. However, the account record may not have a good history of capturing behavioral and context information. Behavioral information that is provided to the account record is related to customer service events (such as when a customer calls for help) and such data is limited. Behavioral and contextual information related to a given customer's use of the offering may be stored in other systems, i.e., it may be fragmented into many separate data stores. Thus, to the health exchange platform, a “customer” is an individual who has a billing account that is associated with an offering and may, or may not, have data on the “employee contacts” that the customer may have made with employees in the call center.

Other elements of data regarding the customer, such as the context of device usage (presence, location, and device type) and other behavioral pattern data (such as which applications are applications used, and when) may be stored or discarded in other systems. Marketing, Product Development, and other teams leveraging specialized analysis software tools can analyze this data.

The health exchange platform logical architecture 100 of the present disclosure provides an improvement over conventional systems that store customer data in separate databases. Such systems create problems, such as different departments and channels have the potential to have contradictory and incomplete “views” of the customer. Data integrity issues proliferate, causing the enterprise to set up teams to manually keep the data in several systems “in sync.” Further, such data is not available in real-time. The present disclosure provides for customer behavior modeling as well as being able to successfully manage a customer through the customer lifecycle, via multiple channels on a single platform.

In conventional systems, data, such as extreme detail records, geo-location, and data usage) are under-leveraged and cannot be practically correlated with a “customer” because a centralized customer view does not exist. If a new product is launched, or a business rule is changed, then at least 22 separate data stores have to be populated with the same master data—causing delays to product rollouts, because the representation of data varies from system to system (depending on its antiquity and propensities of the developers that created the given system). Customers are often presented with inconsistent information (on pricing, features, availability, and eligibility) depending on which channels they use. This means that, depending on which channels a customer uses, customer experience will vary, causing confusion and driving call volumes. For example, if a customer has a stored payment method, it is unlikely that a customer care employee can re-use that data for a customer when the customer wants to process a warranty transaction, which requires that the customer be asked for payment information or similar, multiple times when doing business. Service automation is difficult because of the large number of sub-systems “master data” that need to be coordinated. Such automation is prone to failure if only one of the sub-systems makes a change that “breaks” the workflow.

The health exchange platform logical architecture 100 provides for one object that stores all data associated with a customer, in real-time, and makes such information available to any corporate system that requests appropriate access (that is an “identity service”). Such a common store is structured, secure, and extensible. Thus, individual applications would no longer be tightly coupled to data (by storing data themselves) but become loosely coupled (by leveraging a common data source).

Accordingly, the use of a common “identity service” in the health exchange platform logical architecture 100 provides several beneficial results. For example, all data for a given customer is stored in a single place but also all such data would be structured in a manner that gives it a common meaning across all systems that use customer information. Specifically, master data associated with a customer (such as a definition of eligibility) would be defined in one place. When a new product or service is launched, it would require less time and effort to roll out as the master data is changed in one place. No effort would be required to normalize data, as its meaning would be unambiguous by definition. Behavioral and contextual data will be available in real-time, or near real-time. The health exchange platform has a large scope to define individually customized, content and services across various devices and systems. Common workflows are easier to design. E.g., “Expert-less” sales workflows or workflows that are designed to be relevant to an individual's context and identity. The health exchange platform can more transparently connect subscribers to innovative 3rd party services (“federation”) while maintaining the illusion that the health exchange platform is providing the entire service ecosystem. Due to the ease and low cost of transitioning third-party services in and out of the health exchange platform's offerings, time to market is greatly accelerated, and the health exchange platform can easily experiment with new services, potentially opting to develop the more successful innovations in-house. Further, the health exchange platform improves the information available to front-line employees. Data is consistent, in real-time, individualized to the customer, and is delivered irrespective of the tools used by the employee or the channel to which the employee is affiliated.

In FIG. 1, the presentation layer 106 may be in data communication with the API and communication layer 108 and the business process management layer 110. The API and communication layer 108 may include an application server that contains web services 134, Hypertext Transfer Protocol (HTTP) and interface services 136, a Remote Authentication Dial In Patients Service (RADIUS) server 138, or other Exchanges and programs 140 for implementing application programming interface and communication requirements. In the illustrated implementation, the API and communication layer 108 is implemented as a J2EE application server or enterprise service bus. Those skilled in the art will recognize that the present disclosure is not so limited and the API and communication layer 108 may be implemented in any suitable programming language that allows programming interface and communication capabilities. Examples of other programming languages that may be used to

Implement the API and communication layer 108 including PHP or .NET programming languages. In the illustrated implementation, the API and communication layer 108 may be in data communication with the presentation layer 106, the business process management layer 110, and the data access layer 116.

As shown in FIG. 1, the data management layer 104 may also include a business process management layer 110. In an implementation, the business process management layer 110 may include a data workflow component 142, a business process workflow component 144, a business services component 146, and any other components 148 to implement the business process management layer 110. In the illustrated implementation, the business process management layer 110 is implemented as a Business Process Management (BPM) engine. The BPM engine may be implemented in any suitable programming language capable of handling the components of the business process management layer 110. In an implementation, the business process management layer 110 is in data communication with the presentation layer 106, the API and communication layer 108, and a rules layer 112.

The rules layer 112 may include a data rules component 150, a process rules component 152, and any other components 154 to implement the rules layer 112. The rules layer 112 provides the various rules by which other layers, including the business process management layer 110 and the data access layer 116, may operate or access other Exchanges. The rules layer 112 may be implemented as a rules engine in any suitable programming language capable of providing the necessary control and rules structure for the various layers of the data management layer 104. In an implementation, the rules layer 112 is in data communication with the business process management layer 110 and the data access layer 116.

The data management layer for intent 104 may further include an Identification management layer 114. In an implementation, the management layer 115 may include a credentials provision component 146, a credentials management component 158, a synchronization component 160, and any other components 162 to implement the management layer 115. The ID management layer 114 identifies patients of an Exchange and controls access to resources in the Exchange by placing restrictions on the established identities of individuals or processes. In an implementation, the ID management layer 114 may be implemented as a management tool in any suitable programming language capable of providing credential tracking and access controls. In an implementation, the ID management layer 114 may be in data communication with the data access layer 116 and the LDAP layer 118.

In an implementation, the data management layer 104 may include a data access layer 116. In an implementation, the data access layer 116 may include a data access server 164, Business Access (BI) services 166, batch services 168, and other services 170 to implement the data access layer 116. The data access layer 116 provides access to the various data servers and Exchanges available to the data management layer 104. In FIG. 1, the data access layer 116 is implemented as a J2EE application server. Those skilled in the art will recognize that the present disclosure is not so limited and the data access layer 116 may be implemented in any suitable programming language capable of providing data access capabilities such as, for example, PHP or .NET programming languages. In an implementation, the data access layer 116 may be in data communication with the API and communication layer 108, the rules layer 112, the ID management layer 114, and the database federation layer 120.

The data management layer 104 may also include an LDAP layer 118. In an implementation, the LDAP layer 118 may include an LDAP directory server 172 and LDAP services 174. In the implementation shown in FIG. 1, the LDAP layer 118 is implemented as an open-source LDAP Exchange. Those skilled in the art will recognize that the LDAP layer may be implemented as either an open-source or proprietary LDAP Exchange, and may implement in any suitable programming language. In an implementation, the LDAP layer 118 is in data communication with the ID management layer 114 and the database federation layer 120.

In FIG. 1, the data management layer 104 may include a database federation layer 120. In an implementation, the database federation layer 120 may include an unstructured database 176, a structured database 178, a patient persona database 180, and an operations database 182. The database federation layer may contain any additional databases included in the data management layer 104. The database federation layer 120 may be implemented as an Oracle, NoSQL, or MySQL database server. One skilled in the art will appreciate that the database federation layer may be implemented in any suitable programming language to allow for database access and management by the data management layer 104.

In an implementation, the health exchange platform includes a campaign planning module that provides functions to plan and design healthcare campaigns, as well as manage and monitors campaign delivery activities. By bringing healthcare capability delivery closer to the different groups that make up the healthcare and offer management groups, Telemedicine Providers can support distributed campaign management capabilities through a Single Source of Truth Platform, a health exchange platform.

The health exchange platform is able to integrate several complete competencies. First, Identity and Unstructured Persona of a Patient derives specific behaviors and analytics that will build real-time actionable intent analysis. Greater unstructured analysis can include website visitor, time spent, pages visited statistics as well as cursor movements. Second, Integration into a Health Interaction Platform performs more integrated analysis with our intent analytics engine. Third, Intent Analytics provides reasons for visiting web site, product, service, vacations, destinations, semantic mentions on Telemedicine integrating Telemedicine with web behavior providing a more compelling picture of the patient. Such analytics can allow greater insights for healthcare observing more specifics and insights into brand engagement management. Fourth, the health exchange platform provides Brands the functionality to enable, optimize campaigns and channels by automated call tracking each campaign element through Telemedicine and ePatient Portals.

In various implementations, the health exchange platform provides: present brand channel prioritization and scheduling for complex, multichannel, multistage campaigns; efficient selection, screening and filtering of targeted Telemedicine “influencers” across blogs, social media, API media; coordination and optimization of outbound and inbound communications across multiple channels ensuring that a campaign promotion is not presented differently with Patient Service Agents that Retail Sales Agents or third Party Resellers; the ability to create, deliver and track high-volume, opt-in, personalized e-mail and Telemedicine specific healthcare campaigns; dynamic response handling to automatically update patient contact history, response tracking and analytical analysis; tracking of “hard” responses (purchase decisions) and “soft” responses (subtle changes or trends in Patients behavior), recorded through different channels.

The health exchange platform is able to measure the Impact of Telemedicine digging deeper into the social media subcategories, we see that the average visitor is 65 times more likely to order something than someone coming from either social news or social bookmarking origins. If we break this down even further, using the Key Referrers you can truly identify which site visitors are more likely to buy. This is helpful in planning online media buys.

The health exchange platform can integrate with existing Web Analytics from RCL Website Chat integrating Web Services such as click to chat using VoXML, SIP and XML interfaces. The health exchange platform's ability to merge chat and VoIP signaling together with the ability to capture all meta-data including for healthcare, public relations, sales and patient service. The health exchange platform's Vitality Interaction Platform (SSP) integrates analytics from IoT—Sensors—Patient virtual health channels (EHR, Health Persona) and Web IT Exchanges such as product catalogs and Enterprise Resource Management (ERIP) Exchanges. By creating Patient's “Persona,” the health exchange platform can provide integration with an Identity and Access Management component that builds a real-time ability to understand a prospect or patients past/current experiences.

FIG. 2 shows an example implementation of a Health Intent Engine 200 for creating a Health Persona using normative discourse measures. In the implementation shown in FIG. 2, the listening Post 202 detect data point's 204a-t that correspond to information about a patient from various data sources, including merchandiser websites and Telemedicine websites. The data points 204a-t may include, but are not limited to, Patients 10, Handle, or Screen Name, Name and Network OS, Followers, Friends, or Community, Sentence Structure, Event Based Linguistic, Shares, Likes, Dislikes, or promotes, Brand Loyalty, and Influence Score. The data points 204a-t is analyzed to create a patient persona of patient, which provides information on the patient sentiment, concerns, issues, and trends. In the implementation shown in FIG. 2, the Health Intent Engine 200 is utilizing Linguistics to analyze the language used in messages posted to various social networking sites such as, for example, tweets from Health Persona or status updates from EHR.

In an implementation, the Health Intent Engine 200 in FIG. 2 may analyze one or more analytical indicator(s) 224a-224p. The one or more analytical indicators 224a-224p may be used to determine intent analytics and to derive weighted normative discourse measures including, but not limited to, a weighted context score 226, a sentence context score 228, a tonal score 230, an intonation net score 232, an illocution mean 234, a gross dialect code switch 236, and a Hopi score 238. Using the weighted normative discourse measures, the Intent Engine 200 may provide insight into how patients are reacting to various healthcare and brand engagements and provide opportunities to alter or implement healthcare campaigns in response to patient reactions. Further, the Health Intent Engine 200 may determine one or more scores and/or models, such as scores, measurements and models 240-254, which may be used to determine a patient's health needs.

The competitive landscape may be divided into three sectors and the present disclosure is generally directed to a proactive system of interaction (number three, below):

1. Passive Listening and Reporting—Vendors focused media sourced unstructured data, listening, analytics, management, content, and web and integration.

2. Reactive Data Capture and Analytics—Vendors focused on single and/or multi-channel structured data search, management, and analytics.

3. Proactive System of Interaction—Vendors focused on unstructured and structured data, engagement, personalization and recommendation, and predictive analytics.

In furtherance of the above, the example intent engine 200 of FIG. 2 operates as an unstructured flowing real-time data collection and filtering product that processes health APIs, Cerner API, Epic API, Apple APIs, CRM and Health media dialogue and includes client configuration interfaces based on OAuth II and APIs. In some implementations, the heath exchange platform covers CRM and Health given their high degree of consumer utilization. Additional health and media channels may be accommodated as they become more widely used. The intent engine may be a provides higher orders of patient/customer context leading to superior profiling and targeting; predictive, proactive and automated systems of patient/customer interaction; and demonstrated positive revenue and Health ROI.

To a business function user, the present disclosure provides for improved patient acquisition and retention. The intent engine is able to predict current and patient or patient/customer's likelihood to convert to revenue based on media demand signals.

The present disclosure further provides business benefits by leveraging real-time product, service and event interests to generate net new revenue opportunities. The present disclosure improves patient offer conversion and revenue acquisition through enhanced patient/customer profiling and prioritization based on a patient/customer's interest of promoting their health based on the “outcomes” or goals the patient/customer or patient desires. For example, in Detroit, Mich. a known African American might discuss their need for their growing Diabetes issues and lack of access to insulin because their lack of a “whip.” Under normal considerations and without an Al driven data dictionary to identify key words and phrases as well as “vernacular” including “Intonation” “Code switching” or the speaking constructs that are urban versus professional vernacular drives a Hopi Score. Thus, not only can out app alert her doctor or even possible EMS of a serious situation driven by analytics from her diabetes sensor. Together between streaming real-time information from our APP and sensor driven analytics provides a contextual construct to save a human life. The present disclosure may also reduce costs through more effective media engagement and automation

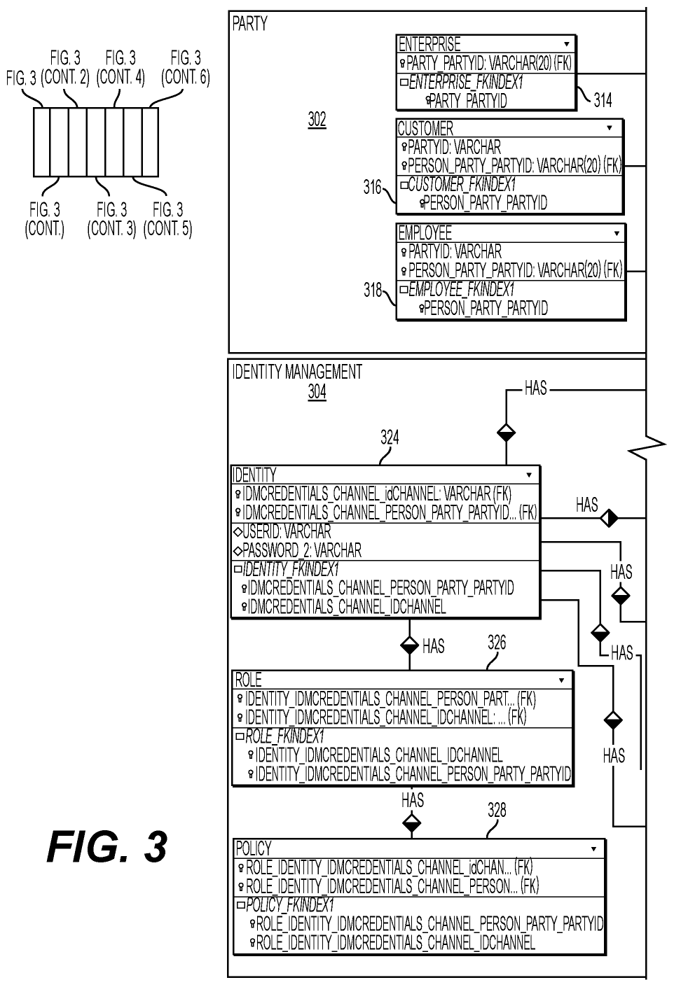

FIG. 3 shows on implementation of a Health Persona reference data model 300. Using key patient identifiers, the SSP builds a profile built on a Service Oriented Architecture (SOA) that creates “Events” for appropriate; healthcare; public relations; sales and patient service interactions. The SSP uses hardened Identity and Access Management (IDAM) principles that uses machine learning to capture the Click to call interaction at the patient level by tying this to a metadata tag or semantic tag with the time, Telemedicine handle, zip code and event code for audit and tracking purposes.

In the implementation shown in FIG. 3, the Health Persona reference data model 300 includes a party component 302, an identity management component 304, a meta persona component 306, a static aspect of persona component 308, and a dynamic aspects of person component 310. Utilizing each of these areas of data, a patient persona may be developed that is tied to patient interactions, providing targeted services or patient interactions. In an implementation, the Health Persona reference data model 300 may tie the meta persona component 306 with time, Telemedicine handle, zip code and event code for audit and tracking purposes.

In an implementation, the party component 302 may include information identifying the enterprise 314, the patient 316, the employee 318, party 320, or person 322. In an implementation, the identity management component 304 includes information regarding identity management credentials 330, an identity 324, a role 326, a policy 328, a certificate 332, a token 334, a security question 336, and a pin 338. In an implementation, the meta persona component 306 may include a persona list 340, a persona context 342, a channel 346, and at least one Telemedicine landscape 348. In an implementation, the static aspects of persona component 308 include a device(s) identifier 350, an address 354, a profile 352, a preference identifier 356, one or more contacts 358, a content component 360, a video component 362, a text component 364, a music component 366, and a photo component 368. In an implementation, the dynamic aspects of persona component 310 include a behavior component 370, an interaction context component 372, a history component 374, an activities component 376, a communication component 378, an e-wallet component, a shopping cart component 382, a message component 384, an events component 386, and a blog component 388.

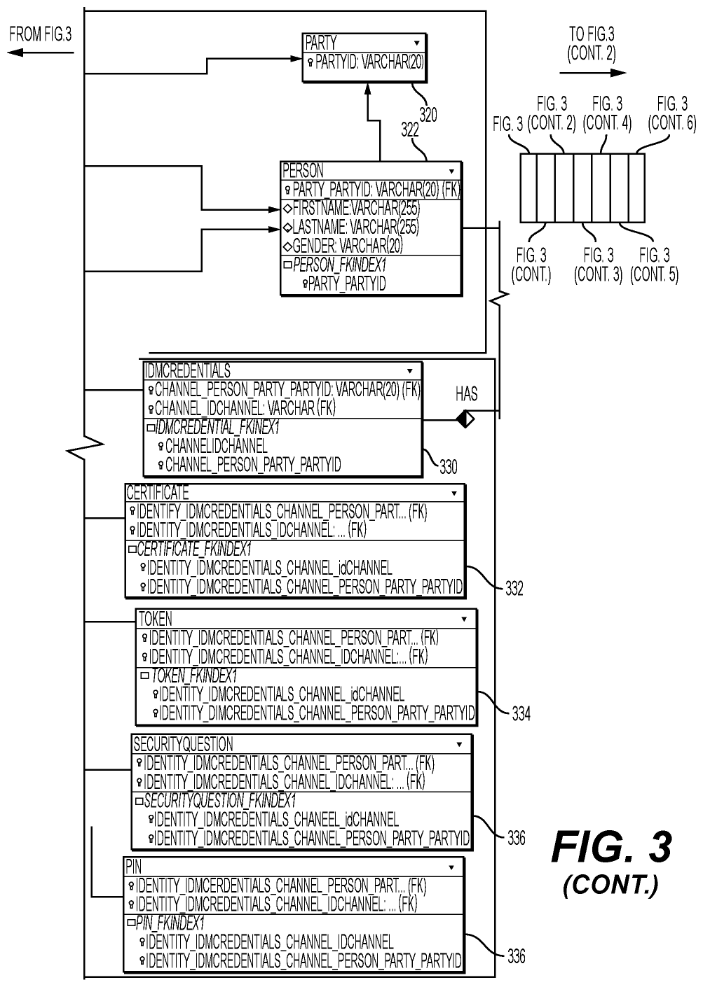

FIG. 4 shows an implementation of Health Persona Interaction with Identity Management. Patients already demand fast, personal, consistent service across multiple channels. Telemedicine means their complaints can reach thousands of people in minutes. This makes it essential to track Telemedicine channels and deploy them as a means to resolve patient grievances and mitigate brand damage. The health exchange platform's packaged technologies can enable a service company to take full advantage of IoT—Sensors—Patient virtual health. They create intimacy with the patient, while automating transactions and interactions, thereby hitting that sweet spot of delivering better service at lower cost. The rich and timely connections made possible by virtual health can increase brand awareness, create a buzz around new products and services, improve patient satisfaction, and build patient loyalty.

In an implementation shown in FIG. 4, a Health Persona interaction 400 is shown. A patient 402 interacts with the health exchange platform Exchange through listening posts and direct interactions with the Patients. From these interactions, a Health Persona Identity 400 is developed that identifies the individual 400. The Health Persona Identity 400 includes a patient identity 406. In an implementation, shown in FIG. 4, the patient identity 406 may include a handle, name, age, address, social security number, credit score, profile(s), OpeniD, and patient value score.

The Health Persona Identity 400 also may include one or more associations with the company 412. In the illustrated implementation, the association with the company 412 includes loyalty ranking, dollars spent, employee event, virtual channel communication, and other physical agent channels (e.g., owned retail, etc.). The associations with the company 412 may be generated by verifying or collection patient identity during interactions. In an implementation, the Health Persona Identity 400 may include an account component 418. The account component 418 may include information relating to identification credentials, account ID, an offering description, a contract description, a device(s) description, or an access to network services (offering). In the illustrated implementation, the Health Persona identity interaction 400 includes one or more associations with third parties 422. The associations with third parties may include agent relationships, virtual channels, social networks known under the trade designation Health Persona/EHR/Scripts, or physical channels (such as owned retail.)

In an implementation, shown in FIG. 4, the individual 402 may have one or more associated context factors 408. In an implementation, the context factors 408 may include presence, check-ins, location, device(s)(s), Telemedicine, influencers KPIs, and affiliations. In an implementation, the unstructured analytics and behavioral information 416 is generated. The unstructured analytics and behavioral information 416 may be generated through various Exchanges and means including, for example, listening posts, intent based algorithms, mobile service usage statistics (voice and data), device(s) usage via Apps, loyalty apps or ePatient, preferences (either explicit or implied), or via Oauth/SAML/OpenlD.

In an implementation, the unstructured analytics and behavioral information 416 is associated with the context factors 408 to generate one or more healthcare offers 414 with which the individual 402 may interact. The healthcare offers 414 may be generated using, for example, Telemedicine, channels, influence score, physician connections, tagged linguistics, device(s) or services, and keywords. In addition to the unstructured analytics and behavioral information 416 and the context factors 408, the Health Persona interaction 400 may leverage an individual's account information 418 to target specific healthcare offers 414. In addition, the healthcare offers 414 may be correlated with specific account information 418.

In an implementation, Health Persona Identity interaction 400 includes one or more files of business intelligence-structured data 428. In the illustrated implementation, the business intelligence structured data 428 may include usage data, behavioral data, device(s) data, context data, or patient contact data. The business intelligence-structured data 428 may interface with the unstructured analytics and behavioral information 416 to store or delete unstructured analytics and behavioral information 416. In an implementation, the business intelligence-structured data 428 may interface with one or more other Exchanges 426. The other Exchange 426 may include demographic data, healthcare data, financial data, network data, manufacturers' data, troubleshooting data, or operational data. In an implementation, the other Exchanges 426 may pole the account information 418 or the business intelligence-structured data 428. In an implementation, the Health Persona interaction 400 may include one hypertext application. The policies 424 may include rule names or algorithms.

The health exchange platform leverages Extensible Markup Language (XML), JavaScript Object Notation (Hypertextplication Programming Interface (API), HyperText Programming (HTTP), Simple Object Access Protocol (SOAP), Representational State Transfer (REST), Oauth, Security Assertions Markup Language (SAML), and OpeniD to support the privacy rights of followers and the current privacy settings on Health Persona and EHR, while adding additional levels of privacy and security. In an implementation, the health exchange platform's productized Social offerings that address specific needs of a Providers' healthcare organization is support of a new product or service launch may include the ability for patients to deliver pre-built campaigns for patient acquisition, development, reactivation and churn management creating brand presence; to facilitate connections, the health exchange platform enable sellers or “Brands” to offer incentives to buyers, such as $5 off a purchase if you follow them on Health Persona or share the product listing with your own followers. The health exchange platform encourages relevant product recommendations to pull demand leading to monetizing patient information-even if you're not looking for something.

In an implementation, the health exchange platform interactions may include social campaign executions. Health campaign execution may include: Permission based controls to ensure that patients are always treated according to their preferences, no matter which channel or touch point is used using Telemedicine Channels with Oauth 2; Healthcare/Sales and Regional Executives can effectively manage the execution of a campaign to adapt to opportunities in their territories, along with coordinated sales communication; Sales Representatives can quickly respond to sales trends and demographic targets with integrated event notifications to ensure that proper approvals are in place to execute real-time against healthcare analytics. Event triggers ensure that campaigns and waves are executed at critical points according to the campaign and reflected in the patient lifecycle; and Inbound and outbound campaign coordination for healthcare control of the interactions that precede or follow a campaign.

FIG. 5 illustrates an example of the health lifecycle of patient with actionable analytics and intent engine 500. A patient lead 502 is acquired from a different healthcare channel or through acquisitions. The patient lead 502 is then used to generate a new patient 504. The new patient 504 will have one or more channels 506 associated with it. Multiple channels in 506 may include pharmacy, retail, Telemedicine, inter-web, blogs, web, commerce, CRM, brokers, APIs or telesales channels. The multiple channels in 506 are used to provide patient data to the communicative intent engine 508. As discussed above, the intent engine 508 uses various analytics to analyze changing patient perceptions, sensors and trends, or to identify reasons for visiting web sites buying products, services, vocations, destination, semantic mentions about virtual health and Telemedicine.

In an implementation in FIG. 5 one or more channels 506 are used to generate one or more patient events 510. The patient events 510 may be based on one or event data structures 514 that can be generated or stored in the Health Exchange database 520. In the illustrated implementation, the patient events 510 are part of patient analytics in 512 are realized by applications. The patient analytics 512 are generated from business functions 524, with the business functions being realized by the patient analytics 512. In an implementation, the business functions 524 may be functional units that support specific business functions 522, such as, for example, retail, care, supply chain, product or service management, finance, ePatient portals, Telemedicine APIs, engineering, service Relationship Risk Management (SRM), healthcare, or billing.

In the example implementations shown in FIGS. 4 and 5, the patient events 510 may serve as inputs to a patient hub implementation approach. The patient events 510, the event data structures 514, and the databases 520 may be inputs into an architecture and design requirement for Session Initiation Protocol (SIP) 516. In an implementation, the architecture and design requirements for SIP 516 may include a business context, one or more data interfaces, service requirements (for example rules, business rules, etc.), services consumption (for example frequency or volume), data stewardship rules, transition of data source requirement (for example gaps, challenges, timelines), and other components to the implement the architecture and design requirements for SIP 516. In an implementation, the enterprise data model is the basis and foundation of the SIP hub.

In FIG. 5, a new patient 504 may also have a persona 518 associated therewith. The persona 518 may be generated based on information from the one or more channels 506. The persona 518 may provide inputs to the communicative intent engine 508 and may be stored in the databases 520. In the illustrated implementation, the new patient 504 may interact with one or more of the patient events 510, transitioning the new patient 504 to an event based patient 526. The event-based patient 526 may then become an active patient 528 during the time that the patient is interacting with the retailer or service provider. Following the active patient 528 portions, the patient then transitions to a post-service patient 530. The post-service patient may be used to generate a new patient lead 502.

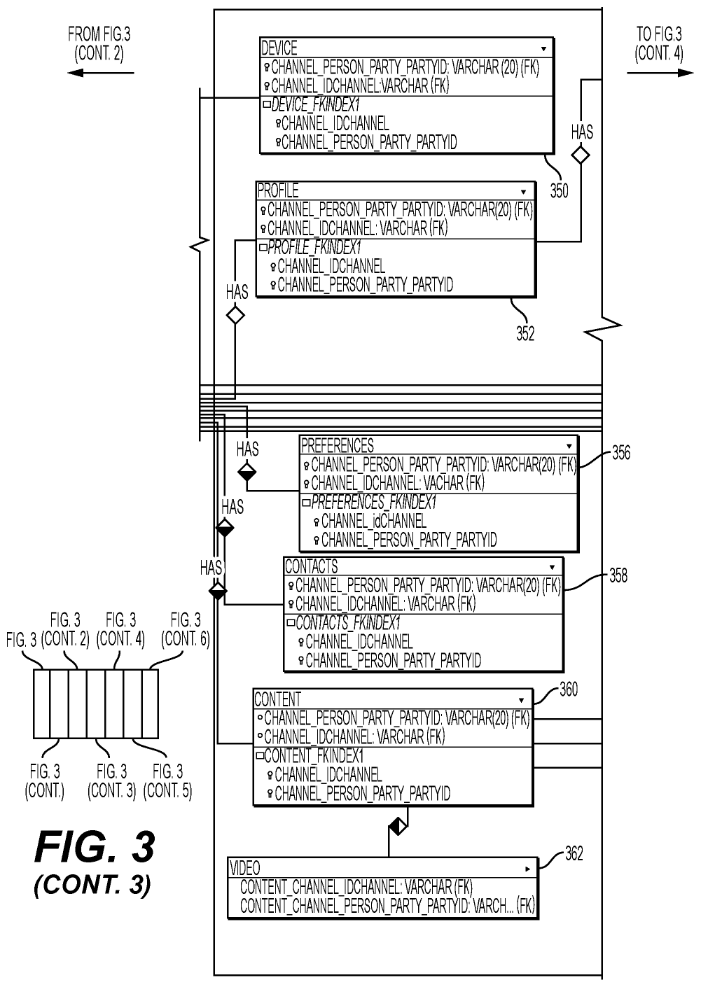

FIG. 6 illustrates an implementation of the health exchange platform Future State SaaS Telemedicine Approach 600. In the illustrated implementation, the health exchange platform lifecycle 602 includes acquiring a lead 606 from a different healthcare channel or through acquisition. The lead 606 may then be transitioned to a new patient 608. The new patient 608 may interact with one or more patient events, causing the new patient 608 to transition to an event based patient 620. The event-based patient is then transitioned to an active patient 622 during the time that the active patient 622 is being serviced by the Patients. Alternatively, the new patient 608 may interact with the patient's business prior to a patient event, and transition directly from a new patient 608 to an active patient 622. Active patients 622 may further interact with patient events, transition between an event based patient 620 and an active patient 622. Once all of the interactions between the active patient 622 and the Patients have been finished, the active patient 622 transitions to a post service patient 624. The post service patient 624 may then be used to generate a new lead 606, such as, for example, in the form of the individual patient, the patient's family, or the patient's corporation

At any point during the health exchange platform lifecycle 602, the patient may interact with the health exchange platform “Exchange” through a patient facing personnel or applications 604. In an implementation, the patient facing personnel or applications 604 may include a web interface, a self-service interface, retail, telesales, a mobile device(s), care, indirect retail, business partners, or other order and service channels. The patient facing personnel or applications 604 interact with the Internet Protocol (IP), JSON, API, 5G, IoT, Sensor Platforms Multimedia System (IMS) platform 618 to allow the patient to interface with the health exchange platform Exchange in a controlled manner. In an implementation, the IMS (IP Multi-media System) or other platform(s) 618 is serviced by the health exchange platform private cloud 610. The health exchange platform private cloud 610 contains the health exchange platform Exchange as discussed above with respect to FIGS. 1-5. The health exchange platform private cloud collects data about the patient for the enterprise analytics engine and to allow the delivery of targeted patient interaction events. In an implementation, a firewall 612 prevents a patient from accessing unauthorized sections of the health exchange platform private cloud 612. In an implementation, the health exchange platform private cloud 610 is serviced by a Software-as-a-Service (SaaS) Patient Relationship Management (CRM) Exchange 616. In the illustrated implementation, the SaaS CRM 616 includes a CRM component, a commission management component, a channel management component, a sales lead management component, a sales analytics component, a patient analytics component, and a leads data federation component. The SaaS CRM 616 may receive inputs from tag based lead information from Health Persona and EHRs.

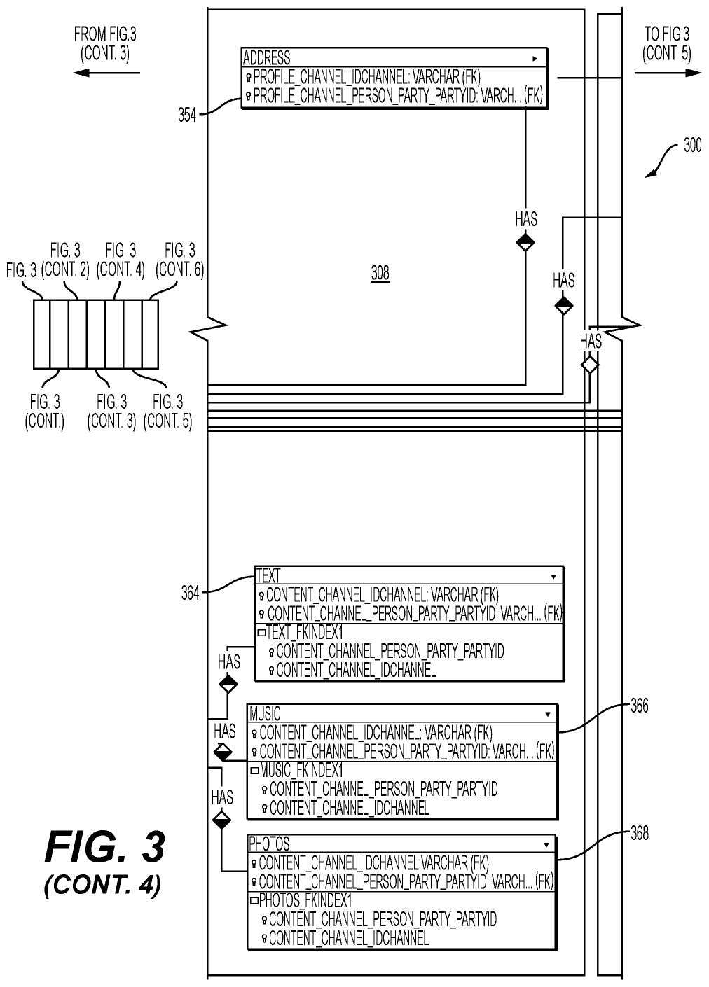

FIG. 7 and the following discussion provide a brief general description of a suitable computing environment in which the described implementations of the health exchange platform Exchange and method may be implemented. It should be understood, however, that handheld, portable, and other computing device(s)s and computing objects of all kinds are contemplated for use in connection with the described implementations. Thus, while a general purpose computing environment is described, this is but one example, and the described implementations may be implemented with other computing device(s), such as a patient having API, network, interoperability, ingestion and interaction. Thus, the described implementations may be implemented in an environment of networked hosted services in which very little or minimal patient resources are implicated, e.g., a networked environment in which the patient device(s) serves merely as an interface to the API, network, bus interoperability, ingestion and interaction, such as an object placed in an appliance, or other computing device(s)s and objects as well. In essence, anywhere that data may be stored or from which data may be retrieved is a desirable, or suitable, environment for operation according to the described implementations.

Moreover, those skilled in the art will appreciate that the described implementations may be practiced with other computer configurations. Other well-known computing Exchanges, environments, and/or configurations that may be suitable for use with the described implementations may include Personal Computers (PCs), server computers, hand-held or laptop device(s)s, multi-processor Exchanges, microprocessor-based Exchanges, programmable patient electronics, network PCs, minicomputers, mainframe computers, mobile computing device(s)s, which may include or be implemented as a combination handheld computer and mobile telephone or smart phone such as a Samsung Galaxy S21 Plus smart phone as well as other types of wireless computing device(s)s having voice and/or data communications functionality such as a handheld device(s), iPAD, mobile telephone, combination mobile telephone, mobile unit, subscriber station, game device(s), messaging device(s), media player(s), IoT device(s), sensor(s) or any other suitable communications device(s) in accordance with the described implementations. The described implementations also may be practiced in distributed computing environments where tasks are performed by remote processing device(s)s that are linked through a communications network/bus or other data transmission medium. In a distributed computing environment, program modules may be located in both local and remote computer storage media including memory storage device(s)s and patient nodes may in turn behave as server nodes.

Although not required, the described implementations can be implemented via an operating Exchange, for use by a developer of services for a device(s) or object, and/or included within application software that operates according to the described implementations. Suitable operating Exchanges include, but are not limited to, operating Exchanges generally known under the trade designation UNIX from the SCO Group, Inc., GHOST for UNIX, MICROSOFT WINDOWS, MAC OS X from Apple Computer, Inc., Internetwork Operating Exchange (IOS) from Cisco, Juniper JUNOS, IBM OS, LINUX, SOLARIS, 3COM, PALM OS (Hewlett Packard), and the like. Software may be described in the general context of computer-executable instructions, such as program modules, being executed by one or more computers, such as patient workstations, servers or other device(s)s. Generally, program modules include routines, programs, objects, components, data structures, and the like that perform particular tasks or implement particular abstract data types. Generally, the functionality of the program modules may be combined or distributed as desired in various implementations.

FIGS. 5, 6 and 7 thus illustrate one example of a suitable computing Exchange 700 in which the described implementations may be implemented. Although as made clear above, the computing environment of the Exchange 700 is only one example of a suitable computing environment and is not intended to suggest any limitation as to the scope of use or functionality of the described implementations. FIG. 7 illustrates an implementation of an Exchange 700. The Exchange 700 may include a communication Exchange having multiple nodes. A node generally may include any physical or logical entity for communicating information in the Exchange 700 and may be implemented as hardware, software, or any combination thereof, as desired for a given set of design parameters or performance constraints. Although FIG. 7 may show a limited number of nodes by way of example, it can be appreciated that additional or fewer nodes may be employed in a given implementation.

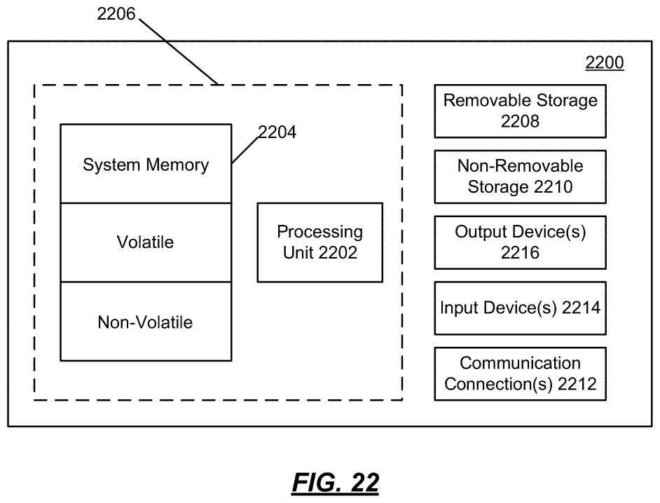

As shown in FIG. 7, the Exchange 700 may be realized as a network 750 including nodes 702 and 704. In various implementations, the nodes 702, 704 may be arranged to operate as computers 710₁₋ₙ and 720₁₋ₙ (where n and m may be any positive integer) connected via the network 750. In an implementation, the computers 710₁₋ₙ, and 720₁₋ₙ, may communicate with the network 750 via a network interface 760, for example. For conciseness and clarity, one example of a suitable general-purpose computing Exchange in which the computers 710₁₋ₙ, and 720₁₋ₙ may be implemented is deferred to the description with reference to FIG. 22 herein below.

As used herein, a node may include any physical or logical entity having a unique address in the Exchange 700. The unique address may include, for example, a network address such as an IP address, a device(s) address such as a Medium Access Control (MAC) address, and so forth. The Patients 790 can access the Exchange from any node, including multiple nodes at the same time. In an implementation, the Exchange 700 may be arranged such that the nodes 702, 704 may be arranged as any one of the computers 710₁₋ₙ and 720₁₋ₙ and may be configured to share the interface 760 to the network 750 (e.g., a LAN interface). In an implementation, any two or more computers 710₁₋ₙ and 720₁₋ₙ may share a single IP address because of limited allocation of IP addresses in the network 750 (e.g., IPv6) or because any two or more computers 710₁₋ₙ and 720₁₋ₙ may likely be accessed using a single IP address or using the same name for the network 750 as though it was a single Exchange, for example.

The nodes 702, 704 of the Exchange 700 may include or form part of the network 750, such as a LAN, a Metropolitan Area Network (MAN), a Wide Area Network {WAN), a Wireless LAN (WLAN), an Internet network, a World Wide Web network, a telephony network (e.g., analog, digital, wired, wireless, Public Switched Telephone Network (PSTN), Integrated Services Digital Network (ISDN) or Digital Subscriber Line (xDSL)), a radio network, a television network, a cable network, a satellite network, and/or any other wired or wireless communications network configured to carry data. The network 750 may include one or more elements, such as, for example, intermediate nodes, proxy servers, firewalls, routers, switches, hubs, adapters, sockets, and wired or wireless data pathways, configured to direct and/or deliver data to other networks.

In an implementation, the nodes 702, 704 may be arranged to operate in accordance with one or more protocols, such as MAC protocols, such as from the IEEE 802.3 series of Ethernet protocols, for example. The nodes 702, 704 may be implemented as a high bandwidth switch, such as a Fast Ethernet switch operating at 700 megabits per second (Mbps), a Gigabit Ethernet switch operating at 1000 Mbps or 10 Gigabits per second (Gbps), a router configured as a DHCP server, and so forth.

The nodes of the Exchange 700 may be arranged to communicate one or more types of information, such as media information and control information. Media information generally may refer to any data representing content meant for a Patients, such as image information, video information. Graphical information, audio information, voice information, textual information, numerical information, alphanumeric symbols, character symbols, and so forth. Control information generally may refer to any data representing commands, instructions or control words meant for an automated Exchange. For example, control information may be used to route media information through an Exchange or instruct a node to process the media information in a certain manner. Other types of information include, without limitation, context information, e.g., information about the Patients 790 such as location, calendar, policy information, logging information, and the like. The information may be communicated from and to a number of different device(s) or networks.

The nodes of the Exchange 700 may communicate in accordance with one or more protocols. A protocol may include a set of predefined rules or instructions to control how the nodes communicate information between each other. The protocol may be defined by one or more protocol standards as promulgated by a standards organization, such as the Internet Engineering Task Force (IETF), International Telecommunications Union (ITU), the Institute of Electrical and Electronics Engineers (IEEE), and so forth. For example, the Exchange 100 may include a packet network communicating information in accordance with one or more packet protocols, such as one or more Internet protocols, including the Transport Control Protocol (TCP) and IP, TCP/IP, X.25, HTTP, Patients Datagram Protocol (UDP), and DHCP protocol. In another example, the Exchange 100 may communicate packets using a medium access control protocol such as Carrier-Sense Multiple Access with Collision Detection (CSMNCD), as defined by one or more IEEE 802.x Ethernet standards. In yet another example, the Exchange 700 may communicate packets in accordance with one or more Asynchronous Transfer Mode (ATM) protocols, Frame Relay, Exchanges Network Architecture (SNA), and so forth. It will be appreciated that the Exchange 700 may communicate packets in accordance with more than one or all of these standards simultaneously.

In various implementations, the Exchange 700 may be illustrated and described as including several separate functional elements, such as modules and/or blocks. Although certain modules and/or blocks may be described by way of example, it can be appreciated that additional or fewer modules and/or blocks may be used and still fall within the scope of the implementations. Further, although various implementations may be described in terms of modules and/or blocks to facilitate description, such modules and/or blocks may be implemented by one or more hardware components (e.g., processors, Digital Signal Processors (DSPs), Programmable Logic Device(s)s (PLDs), Field Programmable Gate Arrays (FPGA), Application Specific Integrated Circuits {ASICs), circuits, registers), software components (e.g., programs, subroutines, logic), and/or combinations thereof.

In various implementations, the Exchange 700 may include multiple modules connected by one or more communications media. Communications media generally may include any medium capable of carrying information signals. For example, communications media may include wired communications media, wireless communications media, or a combination of both, as desired for a given implementation. Examples of wired communications media may include a wire, cable, printed circuit board (PCB), backplane, semiconductor material, twisted-pair wire, co-axial cable, fiber optics, and so forth. An example of a wireless communications media may include portions of a wireless spectrum, such as the radiofrequency (RF) spectrum.

The modules may include, or may be implemented as, one or more Exchanges, sub-Exchanges, device(s)s, components, circuits, logic, programs including computer executable instructions, or any combination thereof, as desired for a given set of design or performance constraints. For example, the modules may include electronic elements fabricated on a substrate. In various implementations, the electronic elements may be fabricated using silicon-based Integrated Circuit (IC) processes such as complementary metal oxide semiconductor (CMOS), bipolar, and bipolar CMOS (BiCMOS) processes, for example.

Some computer applications may include two or more computer Exchanges each including a network management module embedded within a host computer computational platform that share an interface to the network 750. In an implementation, the two or more computer Exchanges may share a single IP address. The two or more computer Exchanges including a network management module may work together or separately, so that when one computer is shut down, hanged, or in standby, the other one is still functional and may be accessed over the network 750 and may operate on behalf of the inoperable computer, for example.

In an implementation, a network management module may include a Network Interface Chip (NIC) that serves a host OS with its own OS driver that includes an embedded manageability module to operate and communicate over network 750 (e.g., LAN, Internet) while the host computer Exchange is operational as well as when the host computer Exchange and its OS are inoperable, disconnected, shutdown, hanged or in standby mode.

In various implementations, the Exchange 700 and methods may be implemented on a variety of Exchanges and device(s). Accordingly, by way example, and not limitation, the process may be implemented as a Patients-controllable learning process in the context of a network firewall, wherein the policy 752 is configured to govern the filtering behavior of the firewall in response to incoming and outgoing packets on the electronic data network 750. As applied in the context of an application level firewall, the policy 752 governs the behavior of the policy Exchange computer 710 (e.g., the policy enforcement computer) in response to traffic on the electronic data network 750 that pertains to specific applications. In other implementations, the process may be applied in the context of an intrusion detection Exchange wherein the policy 752 is configured to determine patterns of traffic on the electronic data network 750 that will or will not be considered indicative of an intrusion. In other implementations, the process may be applied in the context of social networking applications wherein policy 752 is configured to govern the selective disclosure of all types of personal information of the Patients 790. In other implementations, the process may be applied in the context of routing device(s)s on the electronic data network 750 wherein the policy 752 are configured to govern the routing of packets on the electronic data network 750. In other implementations, the process may be applied in the context of home networking device(s)s such as routers, firewalls, and access points wherein the policy 752 is configured to govern the behavior, connectivity, and security of the home networking device(s). In other implementation, the process may be applied in the context of recommender Exchanges for products and services wherein the policy 752 is defined as a model of the preferences, likes, and dislikes of the Patients 190 pertaining to the products and services in question. In other implementation, the process may be applied in the context of software development environments for programming, debugging, and static analysis wherein the policy 752 is defined as a set of patterns within computer programs that represent errors or inefficiencies and a set of proposed improvements to the errors or inefficiencies. In other implementations the process may be applied in the context of email filtering applications wherein the policy is configured to determine which email messages will be deemed to be “spam” or junk emails, “phishing,” or illegitimate solicitation emails, or legitimate emails. The implementation and application contexts described above are not limited in this context.

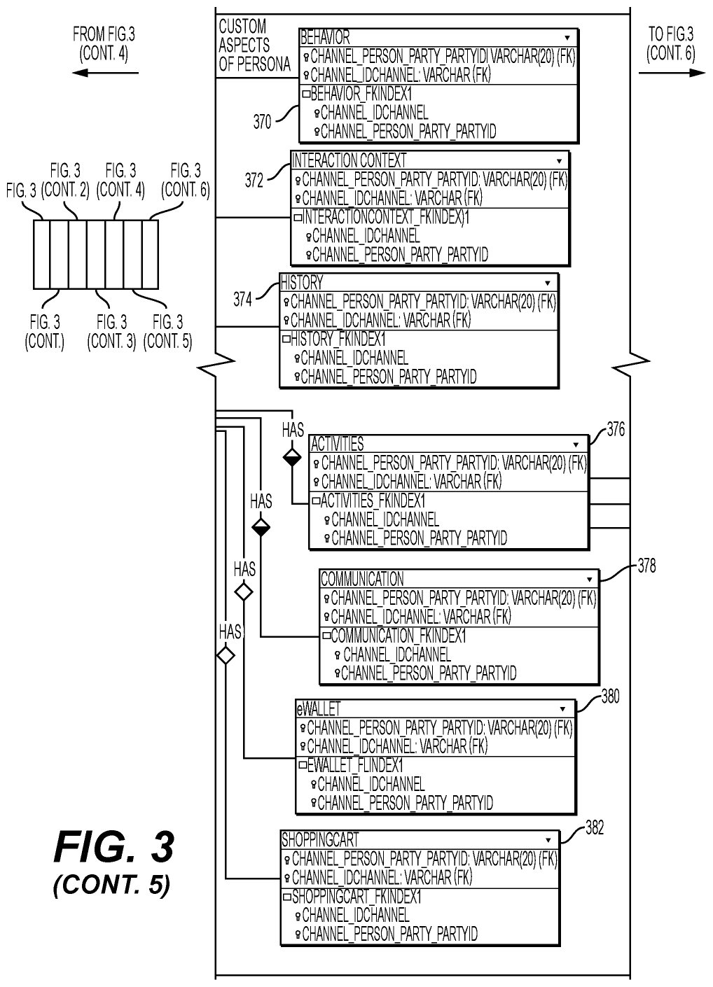

FIG. 8 illustrates an implementation of a patient device(s) interfacing with various Exchanges and partners compatible with the health exchange platform Exchange and method. In an implementation, the patient 826 may use a device(s) 802 to interface with various retailers and websites integrated into the health exchange platform Exchange. The device(s) 802 may be any suitable computing device(s) as described above, such as, for example, a mobile phone. In an implementation, the computing device(s) 802 may include a device(s) identifier 804. The device(s) identifier 804 may include a device(s) type (e.g., PC, phone, etc.) and one or more device(s) capabilities identifiers (e.g., web enable, gee-location enabled, etc.). The device(s) 802 may also include a near-field communication (NFC) component 806 that enables payments at physical retailers using the device(s) 802. In an implementation, the device(s) 802 may also store a patient persona 808 which may include identification attributes, demographic, credentials, behavioral, network, transactions, or presence data. In the illustrated implementation, the device(s) 802 include a session initiation protocol module 812 and an internet data integration module 810.

In an implementation, the patient 826 has one or more patient personas 822 and one or more associated identifying attributes 820. In the illustrated implementation, the associated identifying attributes 820 may be communicated to the device(s) 802 and then communicated further to the health exchange platform Exchange through the device(s) interactions with various retailers and websites.

In the implementation shown in FIG. 8, the patient 826 may interact with various social networking sites 814a-d. As discussed above, the patient's 826 interaction with social networking sites 814a-d may be monitored by a listening post interfaced with the health exchange platform Exchange and the collected data used as part of the analytical engine to determine patient preferences, viewpoints, and trends. In the illustrated implementation, the patient 826 may also interact with one or more channels 824a-d. In an implementation, the patient 826 may interact with a public relations channel 824a, a patient service channel 824b, a healthcare channel 824c, or a Telemedicine channel 824d. As discussed previously, the one or more channels 824a-d may be used to deliver patient interaction events to patients based on the data obtained from the patient profile, listening posts, and the patient's 826 previous interactions with a retailer.

In an implementation, shown in FIG. 8, a patient's 826 may be serviced by a network provider 816 which may provide internet access or other network services. In an implementation, the patient 826 may also interface with partners 818 such as, for example, Amazon.com. The partners 818 may provide various services to the patient through device(s) 802. In an implementation, the services may include product info and reviews, purchasing of goods and services, returns, and patient wish lists.

In an implementation, the health exchange platform Exchange may include a complaint and support scenario. The complaint and support scenario may include identifying top social and networking sites that create relevance drivers including: Technical Constructive Feedback, Unboxing Contributions and Videos, and Contributors with influential followings. The complaint and support scenario may also include capturing information about potential or exiting patients including analyzing contributors and Google.com, WebMD or ClevelandClinic.com recognition for posts and solving problems.

In an implementation, the complaint and support scenario may further include supporting capturing interaction with patient(s) through various channels including: retail sales representatives, patient sales representatives, TeleSales representatives, Telemedicine representatives, and web service and web sales channels. The complaint and support scenario may capture both inbound (initiated by the patient) and outbound (initiated by the Service Provider) requests. In an implementation, the complaint and support scenario may support interaction searches with relevant account level detail and actions taken within an interaction, e.g., problems, account changes and may also support the capability to associate objects such as cases or literature requests with the patient interactions. In an implementation, the complaint and support scenario may include creating an Incident. The incident can be created either manually (out of an interaction) or as a result of a script. Service provider functionality can be leveraged when the incident is created and pushed down to the back-office Trouble Management Exchanges such as Remedy. The complaint and support scenario may support the capability, upon contacting a patient, to have access to the most recent and up-to-date patient and interaction information. In an implementation, the complaint and support scenario may include the ability to create interactions and subsequently, interaction topics such as linking cases to interactions and requests for information. In addition, the complaint and support scenario may include the ability to locate existing patients automatically based on the incoming calls and the ability to create automated and Patients-generated interactions (or memos).

In an implementation, the Exchanges and methods of the health exchange platform may include Patient Branding Management. Patient branding management may include identifying top social and networking sites that create relevance drivers for groups and individuals including: Brand Mentions, Brand Tagging, Negative Posts, and Positive Posts. Patient Branding Management may also include a Person, which is engaged to provide “exclusive access”, or content based on the identity of a group or individual.

In an implementation, patient branding management my include escalating support options for material issues associated with influential persona including listening post logs conversations and analysis tool that detects notable activity. In an implementation, patient-branding management may include acknowledgement and addressing posted issues by having a brand management scenario rate issues and brand management scenario recommends action.

In an implementation, patient-branding management allows a company to keep real time track of brand perception through, for example, logs, rates issues based on a heuristic, measurements of Key Performance Indicators (KPIs), and monitoring brand perception. In an implementation, the patient branding management allows outstanding issues to be mitigated or resolves through relevant engagement. Engagement may be repeatable, predictable, and automated.

In an implementation, patient-branding management provides presentation of facts and data to debunk myths or frauds. Patient branding management may further allow the reuse of patient generated data and provide recognition to such patients by allowing current, interesting and relevant content to be shared.

In an implementation, the Exchanges and method of health exchange platform may include Rebates, Points and Voucher Automation for prescriptions' and IoT device(s)s. Personalization and location allows delivery of services that more closely match the patient's interests. Typical personalization would be enforcing patient communications through the patient preferred communication channel, provide cross-selling and up-selling recommendations based on patient interests and leveraging the information gathered to help the app developer or operator to build a more intimate relationships with the patient.

In an implementation, rebates, points, and voucher automation may include checking in which allows a Patients to collect “points” for checking into stores of interest such as, for example, collecting points just for walking into Publix Pharmacies, Walmart, Health Systems, Hospitals et al. in specific markets such as, for example, New York, Los Angeles, San Francisco, or Chicago to name just a few. Checking-in may also include providing rights to exclusive “points” deals at those stores.

In an implementation, rebates, points, and voucher automation may include scanning, which allows collection of “points” by scanning products in stores with, for example, a mobile phone. Rebates, points, and voucher automation may further include plus incentives, which allow a Patients to look for special promotions for collecting “points.” In an implementation, rebates, points, and voucher automation may include the discovery of Healthcare, Ambulatory Care, Sensors, Prescriptions', Gym Memberships, Co-Pays just to name just a few. Rebates, points, and voucher automation may enable a Patients to share experiences with friends on EHR and Health Persona, and then to stamp a passport to earn rewards at the places that the Patients visits. In an implementation, rebates, points, and voucher automation may allow a Patients to redeem “points” for rewards such as, for example, tablet computers known under the trade designation iPad, music downloads, donations to causes online via websites known under the trade designation Yahoo, Health Persona, Meta, GetUpside, EHR, and mobile carriers such as, for example, AT&T. In an implementation, Rebates, Points, and Voucher Automation may include validating patient satisfaction by validating that predicted/expected value is delivered by the produce/service and that after-sales processes (billing and assurance) are initialized. This process ensures that the patient is satisfied and that the product/service that was actually delivered meets expectations and contracts.

In an implementation, the Exchanges and methods of the health exchange platform include a Sell-Upgrade Patient Offers Automation offering. The Sell-Upgrade-Patient Offers Automation offering may include patient management which allows automation of business process and business flow. In an implementation, the Sell-Upgrade-Patient Offers Automation offering includes integration with mobility applications for campaign management which allows management, automation, and optimization of measurable healthcare campaigns across patient segments, types of campaigns, and healthcare touch points.

In an implementation, the Sell-Upgrade—Patient Offers Automation offering may include Enterprise sales force automation. In various implementations, the Enterprise sales force automation may include the in-store experience, websites, products, or healthcare. The in-store experience may enable shoppers to actively discuss their experiences at brick and mortar locations online. This enables a Patients to find out what patients are saying about, for example, the patient healthcare or Telemedicine shopping experience.

In an implementation, the enterprise sales force automation includes understanding how patients are using a patient's website regardless of whether products are sold online. The website analysis may provide information regarding whether a patient is using the website as an informational or couponing resource. If the website is a transactional health website, Enterprise sales force automation may provide information on what shoppers are saying about, for example, the shipping charges and delivery windows.

In an implementation, the Enterprise sales force automation includes product analysis including the ability to learn how shoppers feel about, for example, a patient's product selection, availability, pricing and quality, as well as private label IoT or pharmaceutical brands. In another implementation, the Enterprise sales force automation may include healthcare information from brand health to community relations health vouchers. In addition, listening to Telemedicine gives retailers the opportunity to gauge awareness of corporate initiatives and healthcare.

In an implementation, the Sell-Upgrade-Patient Offers Automation offering includes Healthcare Offers Automation. In various implementations, Healthcare Offers Automation may include offer responses which are used to tune future offers, Applications logic and corresponding analysis to test and validate, automated creation of offers, and feature re-evaluation for high-impact patients. In an implementation, the Sell—Upgrade—Patient Offers Automation offering includes maintaining Patient call history and handset information for reporting analytical (modeling/segmentation) capabilities. The Sell-Upgrade-Patient Offers Automation offering may further include Enterprise and Small Medium Business Support and services. In another implementation, the Sell-Upgrade-Patient Offers Automation offering may include success evaluation and measurement. In an implementation, the Sell—Upgrade-Patient Offers Automation offering enables patients and partners to integrate the on-demand solution.

The various implementations of offers for the health exchange platform Exchange and method discussed above address key market challenges. The market challenges addressed include, in an implementation, aligning IT services and data to business strategy in an effort to mature IT infrastructure through a process of containment and Rationalization and disentanglement. Technology tools are provided including Listening Posts,IOAM, and Semantic Web Technologies to identify health patterns. The Exchange 700 and method also addresses emphasizing operational security practices to mitigate potential brand risk and provides an appropriate level of confidentiality, integrity, and availability (“security”) for any given solution including identifying reusable services across the enterprise and employing flexible and scalable solutions. The Exchange 700 and method further allows for rightsizing Capex and moving to “pay as you go” Opex models for “development” activities such as Semantic Web and Cloud Computing for Telemedicine.

The various implementations of the health exchange platform and method also allow for integration. The health exchange platform's SSP was built with an understanding of technical IT Integration into Tibco, product catalogs, data marts (Epiphany), IBM Websphere, Oracle Fusion (CRM/OBIEE) and Teradata EDW's. The integration of both structured data and unstructured data with the ability to store/subscribe/publish information such data into existing data warehouses and patient healthcare data marts is critical. Performing analytics in real-time using IoT—Sensors—Patient virtual health and web analytics is cost effective and instantaneous. However, creating and publishing campaigns using structured intelligence to enhance analytics is crucial to a Brands business.

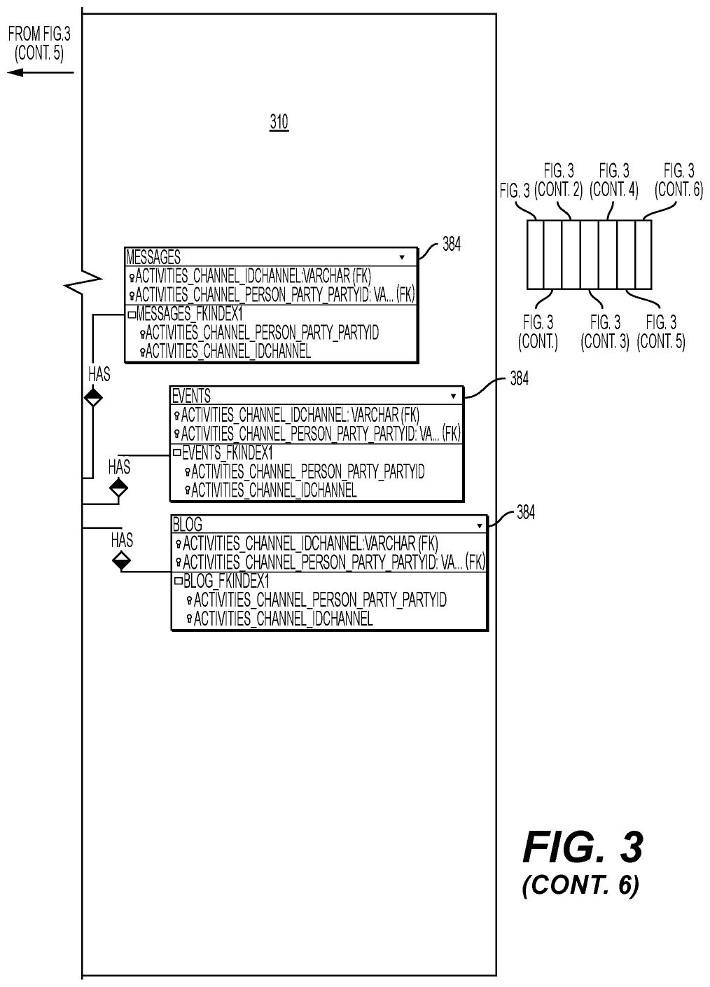

FIG. 9 illustrates example interaction and database attributes to define a patient health persona. A “Persona,” as noted above, is a “360 degree” profile of an individual, including links with other individuals across health APIs, Cerner API, Epic API, Apple APIs, CRM and Health media dialogue. Persona determines and follows an individual's view of a product, brand, or service. Persona also builds networks of Personas to identify the opinion leaders and opinion followers. Actions taken by an individual relative to a product, brand, or service are also tracked. Using tools such as OAuth, OpenID, and SAML, Persona determines when two or more individuals might actually be the same person. Through integration with a client CRM system Persona will link Personas to specific patient/customers based on captured information, actions taken, etc. Analytics provide for a customizable monitoring, analysis, and reporting tool that captures streams of data from the entire Platform to perform. Analytics provides real-time dashboard based activity reporting, delivery of standard summary & detail reports, and delivery of custom reports.

### EXAMPLE

Persona WS.gatherhealthUserProfiles

Calls healthAction.gatherUserProfiles

healthAction. gatherUserProfiles
gets records from users table where user_name is null to get health user information
gets a maximum of 14999 users, since we can get 100 *150=15000 users per cycle i.e., per 1 hour before we hit Health API limit.
does health lookupUsers API call passing in a max. of 100 users per request.
updates health_user table with user information
adds a record to persona table with handleType=“Health”

ListenWS.gatherCRMUserProfiles

calls CRMAction.gatherUserProfiles

CRMAction. gatherUserProfiles
gets all records from CRM_users table where first_name is null to get CRM user information
does CRM fetchObject API call with CRM user id & “User.class” as input parameters
updates CRM_user table with user information
adds a record to persona table with handletype=“CRM

### Example User Interfaces

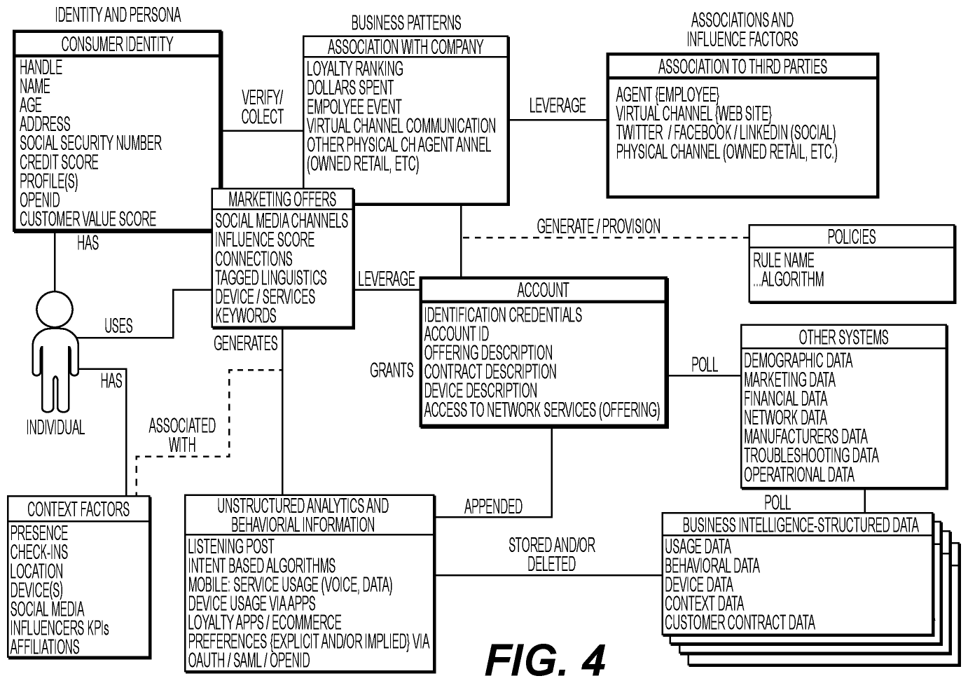

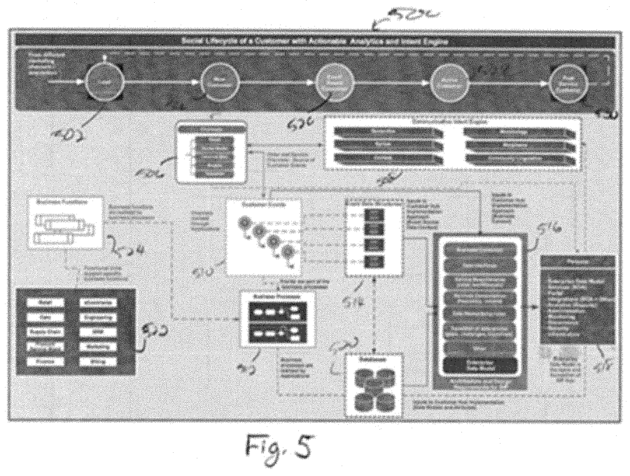

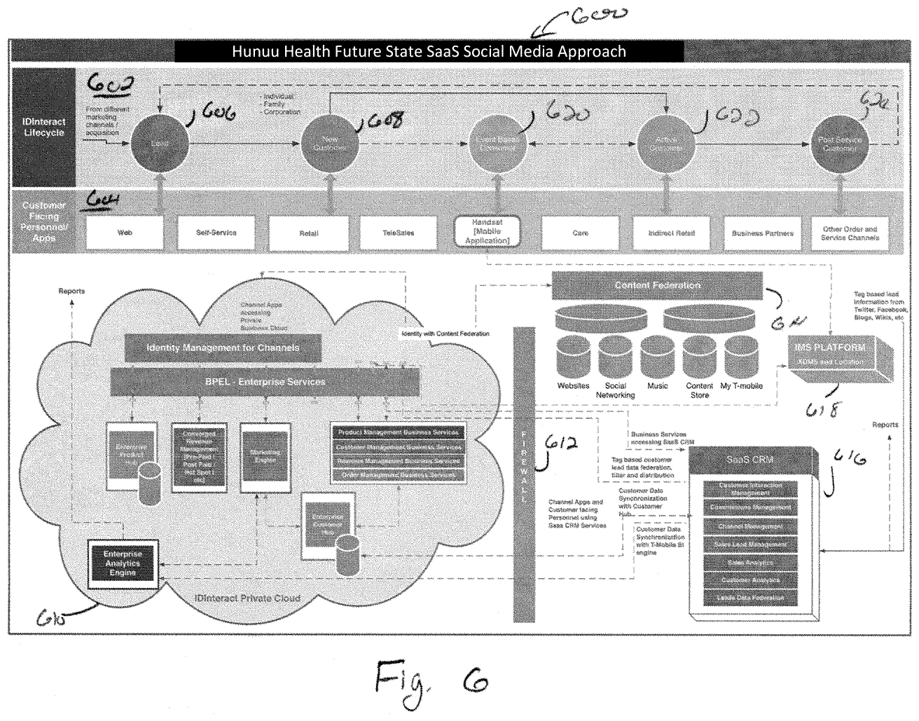

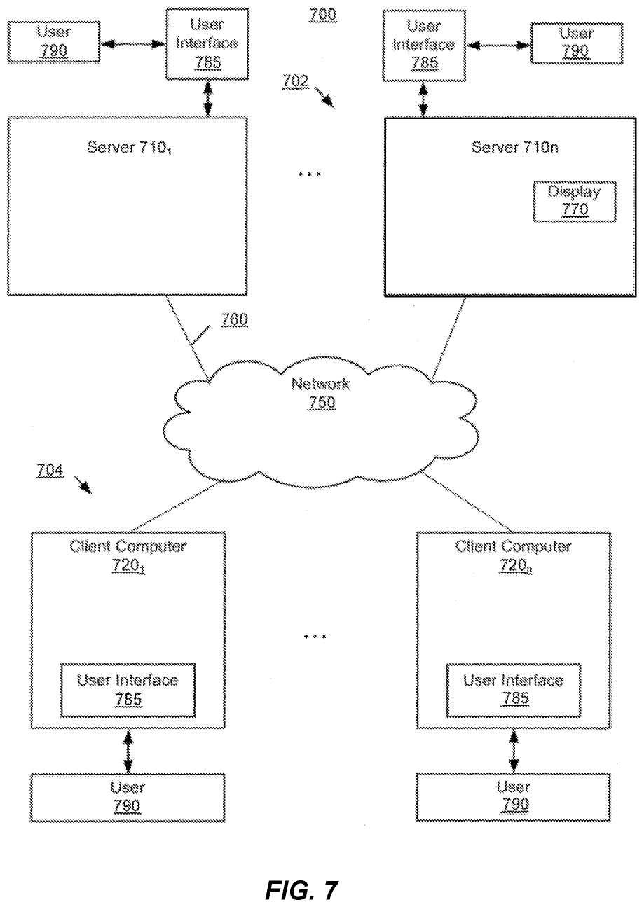

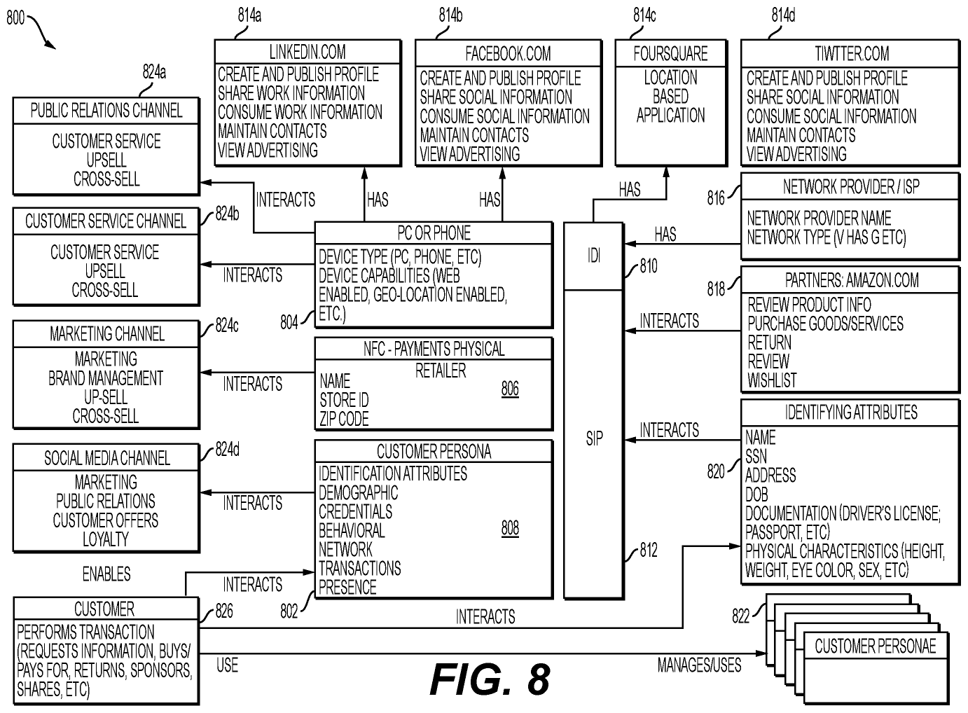

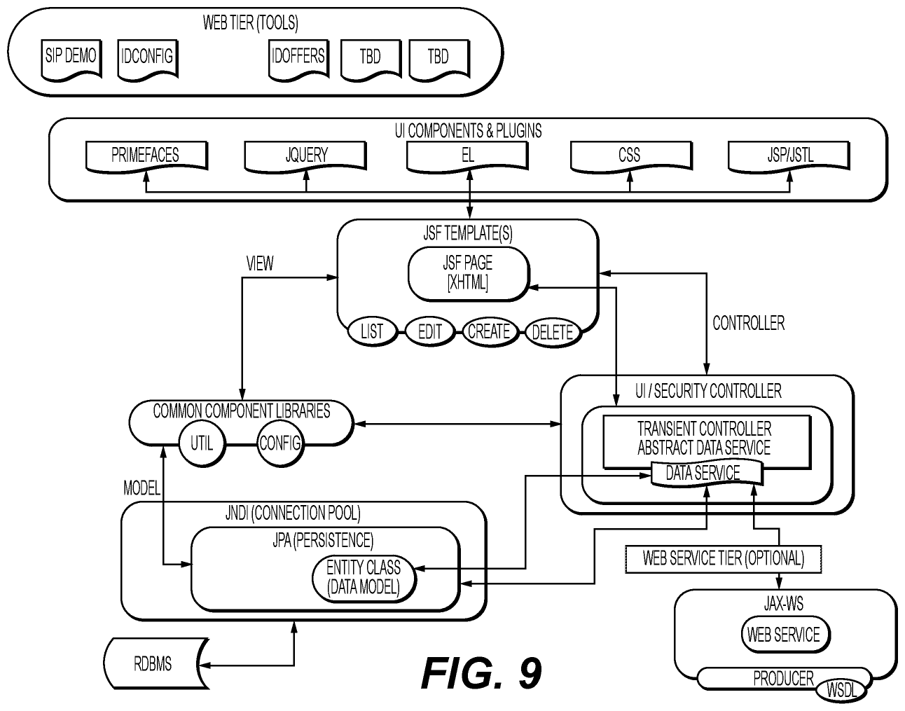

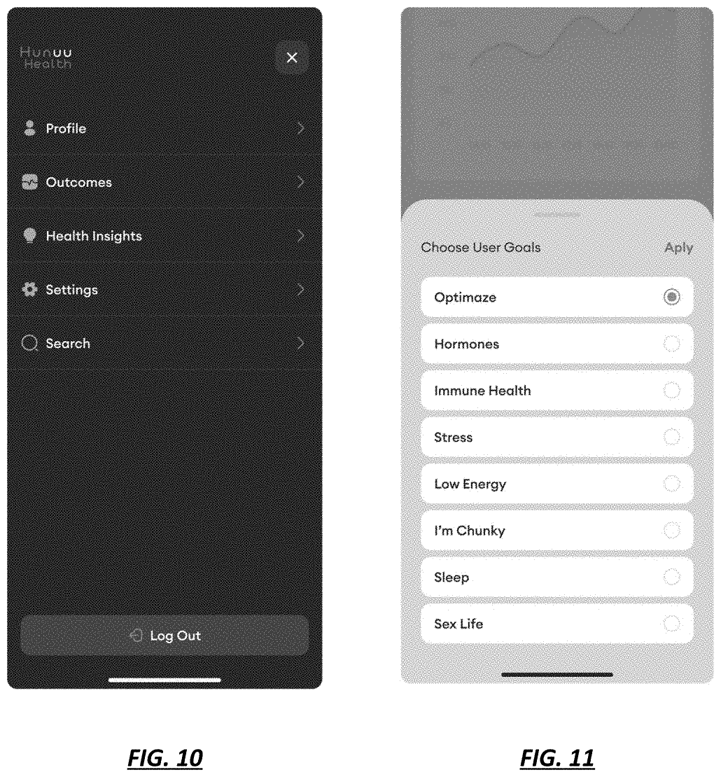

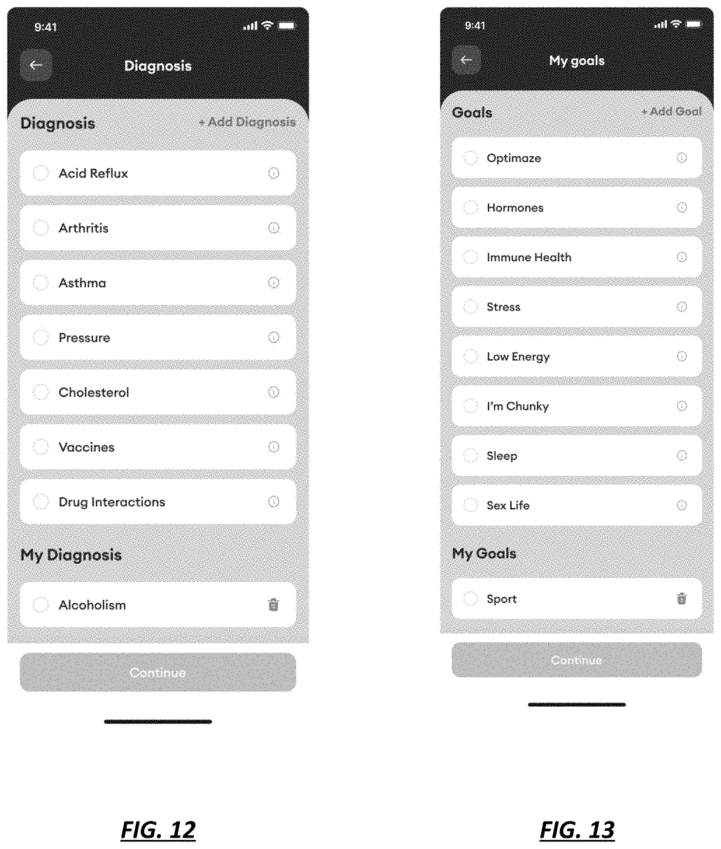

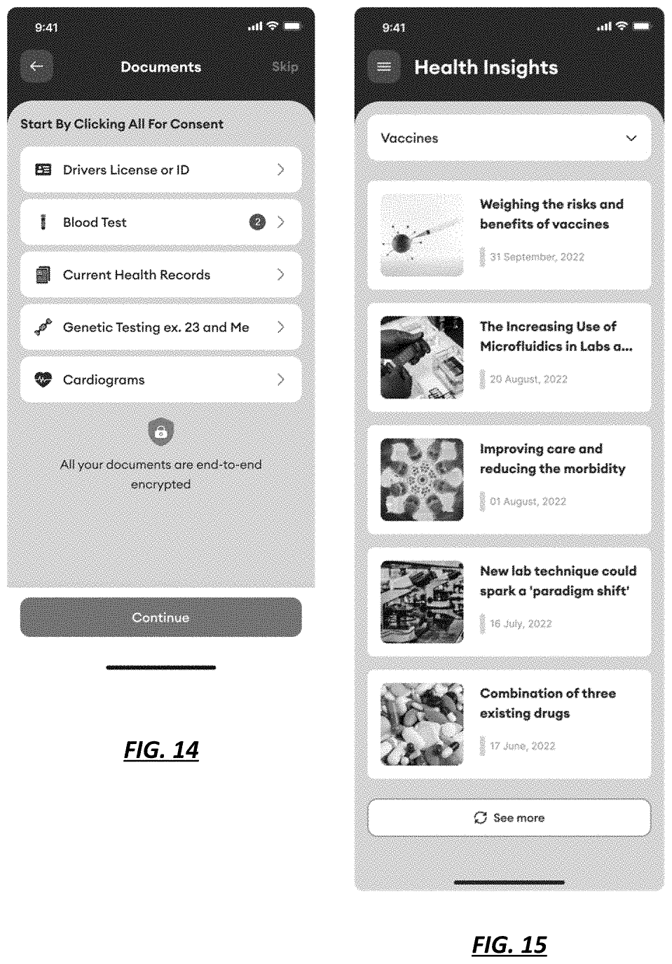

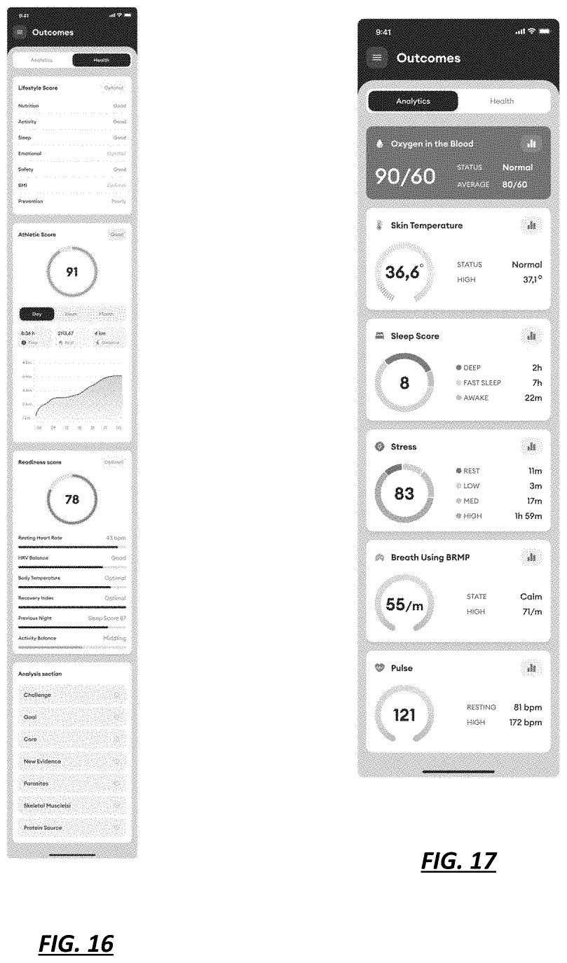

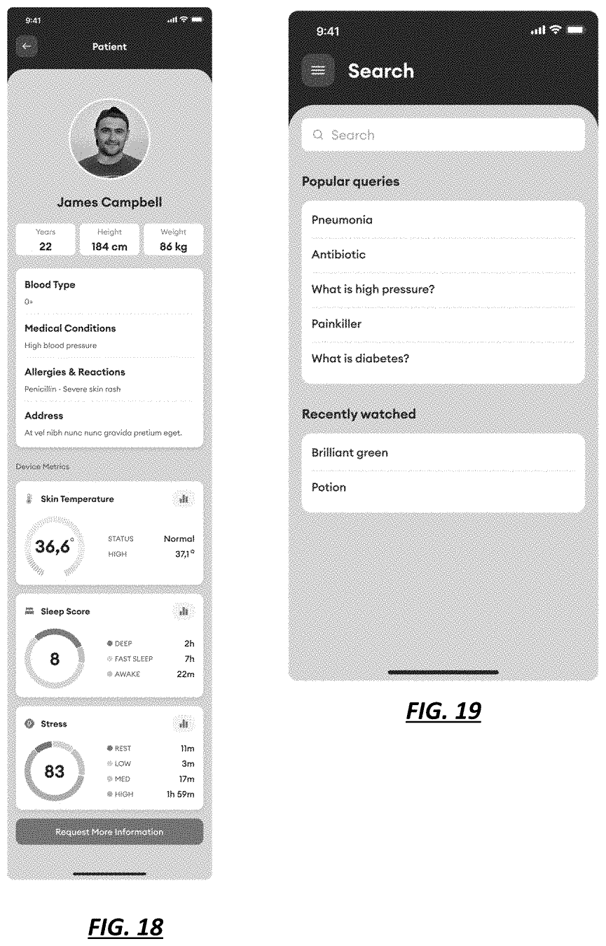

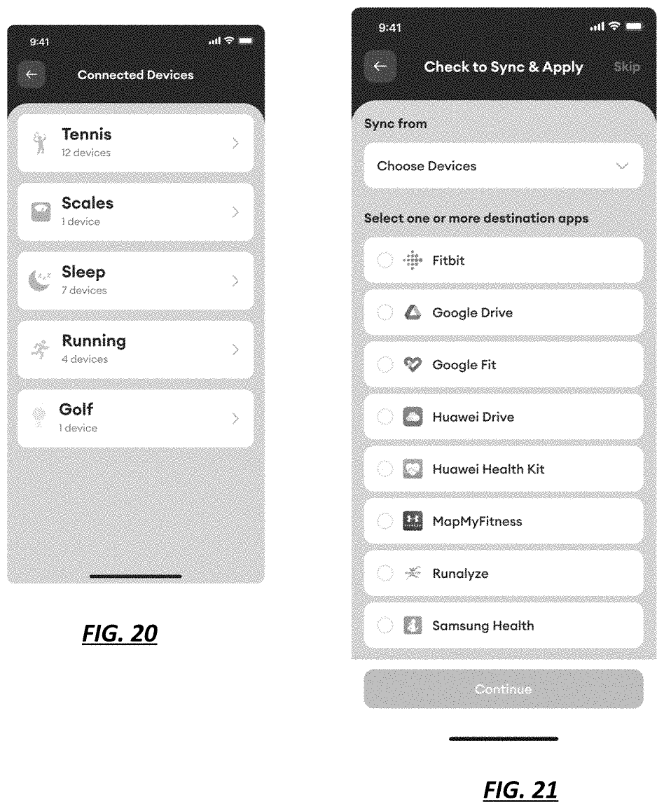

FIGS. 10-21 illustrate example user interfaces that may be incorporated into the health exchange platform of the present disclosure. FIG. 10 illustrates an example user interface wherein a patient may set profile information, outcomes, search for health insights and change user settings. FIG. 11 illustrates an example user interface wherein a patent may set user goals. FIG. 12 illustrates an example user interface wherein a patient may search for a diagnosis based on symptoms provided to the health exchange platform. FIG. 13 illustrates an example user interface wherein a patient may set goals. FIG. 14 illustrates an example user interface wherein a patent may upload, view and/or edit documents stored in the health exchange platform. FIG. 15 illustrates an example user interface wherein a patent may review information (health insights) based on entered search criteria. FIG. 16 illustrates an example user interface wherein a patent may see a summary of health data outcomes. FIG. 17 illustrates an example user interface wherein a patent may view analytics about physiological conditions. FIG. 18 illustrates an example user interface displaying patient data and information. FIG. 19 illustrates an example search user interface. FIG. 20 illustrates an example user interface that displays user devices that are connected to the health exchange platform. The user devices may provide data to the health exchange platform to determine outcomes. FIG. 21 illustrates an example user interface wherein devices may be selected such that data may be provided to the health exchange platform.

### Example Patient/Customer Engagement Scenarios

The methods and systems of the present disclosure may be used to drive traffic into online telehealth and ambulatory locations based on seasonal events (e.g., fall versus winter) or Identify patient/customers with high event interest and opportunity revenue. They may also provide information to influence and accelerate the purchase decision or extend an offer or promotion to incent the purchase decision. The methods and systems may accelerate patient/customer awareness, consideration and purchase for online and in-store campaigns, identify patient/customers with high campaign interest and opportunity revenue, provide information to influence and accelerate the purchase decision, and extend an offer or promotion to incent the purchase decision

Implementations of the present disclosure may contact patient/customers to nurture and close product or service purchase decisions, provide information to influence and accelerate the purchase decision, extend an offer or promotion to incent the purchase decision, and extend an offer to provide immediate patient/customer contact to answer questions and assist with the purchase decision Some implementations may contact groups to nurture and close product or service purchase decisions, provide information to influence and accelerate the purchase decision, to extend an offer or promotion to incent the purchase decision, and extend an offer to provide immediate patient/customer contact to answer questions and assist with the purchase decision.

In other aspects, the systems and methods of the present disclosure automate client/IDI processes. For example, a client communicates to the desired target audience and patient/customer engagement objectives through the APIs and driven by the Intent Platform. The health exchange platform provides an understanding of patient/customer interest and influence linked to geography, events, and promotions through data collection, analysis, and categorization relative to desired patient/customer engagement(s) (e.g., Company/Brand Name, Product/Service Name, Information Query/Search/Subject, Patient/customer Demand Signals, Patient/customer Influence Rating (Followers), Patient/customer Linguistics (Conversational items, statement, questions), Demographics (Male, Female, Age Ranges), Patient/customer Emotions (Happy, Sad, Angry, Satisfied, Complacent), Patient/customer Intents and Actions (Buy, Bought, Return, Recommend, Reviewed), and/or Patient/customer Comments (Positive, Negative). In yet other aspects, the systems and methods of the present disclosure configure a client User Interface (UI) and Interaction Platform to present and enable execution of the desired patient/customer engagement(s) using the designated client CRM system(s).

In other aspects, the systems and methods of the present disclosure configure a client UI and Social Interaction Platform (SIP) to provide the desired measurement and reporting, such as number and type of offers issued, number of responses to offers issued, number of offers outstanding, number of sales opportunities from offers issued, patient/customer trends (emotion dimension), and influencer trends (top influencers, followers). The system and methods further optionally configures the SIP for data integration with existing CRM systems, product catalog, MDM sources, patient and customer identifier, billing systems, web/e-commerce systems, BI/EDW and cloud. The IDI configures the SIP to search and compile specific target audience personas (i.e., patient/customer contacts) from measured media channels, such as APPLE, GOOGLE, GARMIN, TWITTER, FACEBOOK, LINKEDIN, PINTEREST, MEDRX, forums, blogs and/or news. The health exchange platform may further optionally enhance client personas with data integration from existing client CRM system(s) by integrating patient/customer status, preferred channel (store, online, other), online user IDs, phone number, email address, physical address, loyalty program ID, health insurance, loyalty reward status, total revenue/year (value), health purchases, HOPI score, and/or a lifetime value score. The health exchange platform may conduct predictive and linguistic data analytics to refine and categorize the personas by desired patient/customer engagement(s). The health exchange platform enables execution of the desired patient/customer engagement(s) using the client UI by associating the personas to the [symbol] appropriate interaction capability for the patient and/or client designated CRM system(s). Yet further, the health exchange platform may provide client documentation, training, and support for UI access and utilization.

Aspects of the present disclosure may automate client/patient processes. Here, a client communicates the desired target audience and audience engagement objectives to IDI in a briefing. The health exchange platform provides understanding of audience interest linked to geography, events, and promotions through data collection, analysis, and categorization relative to desired audience engagement(s). for example, the following may be collected: company/brand name, product/service name, information query/search/subject, audience demand signals, audience linguistics (conversational items, statement, questions, demographics (male, female, age ranges), audience emotions (happy, sad, angry, satisfied, complacent), audience intents and actions (buy, bought, return, recommend, reviewed), and/or audience comments (positive, negative). The IDI configures the client User Interface (UI) and Social Interaction Platform (SIP) to present and enable execution of the desired audience engagement(s) using designated client CRM system(s). The IDI configures the client UI and Social Interaction Platform (SIP) to provide the desired measurement and reporting, such as number and type of audience engagements initiated, number of responses to initial audience engagements, number of initial audience engagements outstanding, audience trends (emotion dimension) and/or influencer trends (top influencers, followers). The platform may configure data integration with existing CRM systems (optional): product catalog, MDM sources, customer identifier, billing systems, web/e-commerce systems, BI/EDW. The health exchange platform optionally enhances the client personas with data integration from existing client CRM system(s) by integrating an audience contact status (y/n), preferred channel (store, online, other, online user id (y/n), phone number, email address, physical address, loyalty program id, loyalty reward status, total revenue/year (value), product purchases, and/or lifetime value score. The health exchange platform conducts predictive and linguistic data analytics to refine and categorize the personas by desired audience engagement(s). The health exchange platform enables execution of the desired audience engagement(s) using the client UI by associating the personas to the [symbol] appropriate interaction capability and/or client designated CRM system(s). The health exchange platform provides client documentation, training, and support for UI access and utilization.

Further aspects of the present disclosure may automate patient processes. For example, a client communicates the desired target audience and audience engagement objectives to IDI in a briefing. The IDI provides understanding of audience interest linked to geography, events, and promotions through data collection, analysis, and categorization relative to desired audience engagement(s). This may include company/brand name, product/service name, information query/search/subject, audience demand signals, audience linguistics (conversational items, statement, questions), demographics (male, female, age ranges), audience emotions (happy, sad, angry, satisfied, complacent), audience intents and actions (buy, bought, return, recommend, reviewed), and/or audience comments (positive, negative). The health exchange platform configures the client User Interface (UI) and Health Interaction Platform (HIP) to present and enable execution of the desired audience engagement(s) using the HIP or designated client CRM system(s). The health exchange platform configures the client UI and Health Platform to provide the desired measurement and reporting. For example, this may include a type of audience engagements initiated, number of responses to initial audience engagements, a number of initial audience engagements outstanding, audience trends (emotion dimension), and/or influencer trends (top influencers, followers). The health exchange platform may optionally configure data integration with existing CRM systems, such as product catalogs, MDM Sources, customer identifiers, billing systems, Web/e-Commerce Systems, and/or BI/EDI.

Further aspects of the present disclosure may identify marketplace trends. Here, the IDI configures the Social Interaction Platform (SIP) to search and compile specific target audience Personas (i.e., discreet contacts) from measured social media channels, such as those noted above. The IDI optionally enhances the client personas with data integration from existing client CRM system(s). This may include audience contact status (y/n), preferred channel (store, online, other, online user id (y/n), phone number, email address, physical address, loyalty program id, loyalty reward status, total revenue/year (value), product purchases, and/or lifetime value score. The IDI conducts predictive and linguistic data analytics to refine and categorize the Personas by desired audience engagement(s). The IDI enables execution of the desired audience engagement(s) using the client UI by associating the Personas to the [symbol] appropriate IDI social interaction capability and/or client designated CRM system(s). The IDI provides client documentation, training, and support for UI access and utilization.

In accordance with yet further aspects of the present disclosure, the health exchange platform my perform the following functions:

1. Ingest rich data sources—at the right frequency and data latency to Handle multiple ingest points to deal with both streaming and batch processing which can be accomplished with the following open source architectures including: Kylo.io; Spark to orchestrate ingestion and processing.

2. Ingest self-service data streams into real-time and near real-time ACID databases including: Web page activity; IoT/Sensors; Virtual health Streams, Electronic Health Records, CRM Systems, Prescription Systems

3. Provide a window into the development of integrated exchange metrics including: Metadata; usage Information; search capabilities; sharing tool(s); streaming data sources of record; Integration into University

The health exchange platform connects and aggregates structured, unstructured and outcome based or derivative data; and builds relationships and connections between geo-location, hospital exchanges, providers, insurers and third party add-on insurance like travel, disability, etc. The health exchange platform integrate the data using the customer identity register and standardizing customer interactions. In one aspect, the present disclosure is directed to a predictive analytics engine as the health exchange platform makes it easier for patients to visualize and securely store their health records. Patients can aggregate health records from multiple providers alongside personal generated data, creating a more holistic predictive view of health outcomes. Transactional ID Reconciliation Events are tied to a Customer Record and create an expanded single customer view: To resolve known and unknown customer identities; to provide conformed customer dimension to downstream applications. The above includes a number of attributes, including, but not limited to: metadata capture, data access, data governance, data cleansing, data stewardship, reusable workflows, support of real-time workflows and hybrid architecture(s).

As now is evident from the above description, the health exchange platform further connects predictive health results to health care providers, health exchanges and/or insurance companies. The health exchange platform deploys “health behaviors” that combines IOT sensor(s), EHRs, analytics, prescriptions', sexual health data, training data, physiological elements with artificial intelligence (AI) based on exercise and DNA; combines real-time health patterns with genetic pharmacological data identifying medical interactions, and is a SaaS platform that integrates and standardizes wearable and sensor analytics using machine learning (NL)

The health exchange platform may also perform functions a “health advocate” by providing access, via an app, to a single source of truth for all historical and real-time data/health events/sensors to improve health outcomes with physicians/dentists/specialists. The health exchange platform includes predictive intelligence that employs Al together with real-time integration with a patient's sensors to supply predictive outcomes based on health records (EHRs), historical health data, sensor intelligence, DNA testing, blood tests, vaccinations, past surgeries and current health conditions. The single source of truth creates “health outcomes,” which frees personal data that is siloed in proprietary apps, thus creating security to a patient to know and own his/her own health data.

Below is a non-limiting list of data that may be ingested by the health exchange platform that may be used to perform the functionalities described above:

DNA
Age-Related Macular Degeneration Variant detected, not likely at increased risk
Celiac Disease Slightly increased risk
Irritable Bowel Syndrome-Plus Increased likelihood
Kidney Stones-Plus Increased likelihood
Late-Onset Alzheimer's Disease Slightly increased risk
Migraine-Plus Increased likelihood
Psoriasis-Plus Increased likelihood
Severe Acne-Plus Increased likelihood
Alpha-1 Antitrypsin Deficiency Variants not detected
Atrial Fibrillation-Plus Typical likelihood
BRCA1/BRCA2 (Selected Variants) Variants not detected
Chronic Kidney Disease (APOL1-Related) Variants not detected
Coronary Artery Disease-Plus Typical likelihood
Diverticulitis-Plus Typical likelihood
Eczema (Atopic Dermatitis)-Plus Typical likelihood
Familial Hypercholesterolemia Variants not detected
G6PD Deficiency Variants not detected
Gallstones-Plus Typical likelihood
Gestational Diabetes-Plus Typical likelihood
Glaucoma-Plus Typical likelihood
Gout-Plus Typical likelihood
HDL Cholesterol-Plus Typical likelihood
Hereditary Amyloidosis (TTR-Related) Variants not detected
Hereditary Hemochromatosis (HFE Related) Variants not detected
Hereditary Thrombophilia Variants not detected
High Blood Pressure-Plus Typical likelihood
LDL Cholesterol-Plus Typical likelihood
MUTYH-Associated Polyposis Variants not detected
Nonalcoholic Fatty Liver Disease-Plus Typical likelihood
Obstructive Sleep Apnea-Plus Typical likelihood
Parkinson's Disease Variants not detected
Polycystic Ovary Syndrome-Plus Typical likelihood
Restless Legs Syndrome-Plus Typical likelihood
Skin Cancer (Basal and Squamous Cell Carcinomas)-Plus Typical likelihood
Skin Cancer (Melanoma)-Plus Typical likelihood
Triglycerides-Plus Typical likelihood
Type 2 Diabetes Typical likelihood
Uterine Fibroids-Plus Typical likelihood

Pharmacogenetics
CYP2C19 Drug Metabolism-Plus Predicted intermediate metabolizer
Related Medication Insightsfor Cyp2C19 Drug Metabolism
Citalopram)(Celexa®)-Plus View Report
Clopidogrel)(Plavix®)-Plus View Report
DPYD Drug Metabolism-Plus Predicted normal metabolizer
SLCO1B1 Drug Transport-Plus Predicted normal function

Carrier Status
ARSACSVariant not detected
Agenesis of the Corpus Callosum with Peripheral Neuropathy Variant not
detected
Autosomal Recessive Polycystic Kidney Disease Variant not detected
Beta Thalassemia and Related Hemoglobinopathies Variant not detected
Bloom Syndrome Variant not detected
Canavan Disease Variant not detected
Congenital Disorder of Glycosylation Type 1a (PMM2-CDG)Variant not detected
Cystic Fibrosis Variant not detected
D-Bifunctional Protein Deficiency Variant not detected
Dihydrolipoamide Dehydrogenase Deficiency Variant not detected
Familial Dysautonomia Variant not detected
Familial Hyperinsulinism (ABCC8-Related)Variant not detected
Familial Mediterranean FeverVariant not detected
Fanconi Anemia Group CVariant not detected
GRACILE SyndromeVariant not detected
Gaucher Disease Type 1Variant not detected
Glycogen Storage Disease Type laVariant not detected
Glycogen Storage Disease Type IbVariant not detected
Hereditary Fructose IntoleranceVariant not detected
Herlitz Junctional Epidermolysis Bullosa (LAMB3-Related)Variant not detected
Leigh Syndrome, French Canadian TypeVariant not detected
Limb-Girdle Muscular Dystrophy Type 2DVariant not detected
Limb-Girdle Muscular Dystrophy Type 2EVariant not detected
Limb-Girdle Muscular Dystrophy Type 2IVariant not detected
MCAD DeficiencyVariant not detected
Maple Syrup Urine Disease Type 1BVariant not detected
Mucolipidosis Type IVVariant not detected
Neuronal Ceroid Lipofuscinosis (CLN5-Related)Variant not detected
Neuronal Ceroid Lipofuscinosis (PPT1-Related)Variant not detected
Niemann-Pick Disease Type AVariant not detected
Nijmegen Breakage SyndromeVariant not detected
Nonsyndromic Hearing Loss and Deafness, DFNB1 (GJB2-Related)Variant not
detected
Pendred Syndrome and DFNB4 Hearing Loss (SLC26A4-Related)Variant not
detected
Phenylketonuria and Related DisordersVariant not detected
Primary Hyperoxaluria Type 2Variant not detected
Pyruvate Kinase DeficiencyVariant not detected
Rhizomelic Chondrodysplasia Punctata Type 1Variant not detected
Salla DiseaseVariant not detected
Sickle Cell AnemiaVariant not detected
Sjögren-Larsson SyndromeVariant not detected
Tay-Sachs DiseaseVariant not detected
Tyrosinemia Type (Variant not detected
Usher Syndrome Type 1FVariant not detected
Usher Syndrome Type 3AVariant not detected
Zellweger Syndrome Spectrum (PEX1-Related) Variant not detected

Wellness
Alcohol Flush ReactionUnlikely to flush
Caffeine ConsumptionLikely to consume more
Cat Allergy-Plus Typical likelihood
Deep Sleepless likely to be a deep sleeper
Dog Allergy-Plus Typical likelihood
Genetic Weight Predisposed to weigh less than average
Lactose Intolerance Likely tolerant
Muscle Composition Common in elite power athletes
Nearsightedness-Plus Typical likelihood
Saturated Fat and Weight Likely similar weight
Sleep Movement Likely more than average movement

With reference to FIG. 22, an exemplary system for implementing aspects described herein includes a computing device, such as computing device 2200. In its most basic configuration, computing device 2200 typically includes at least one processing unit 2202 and memory 2204. Depending on the exact configuration and type of computing device, memory 2204 may be volatile (such as random access memory (RAM)), non-volatile (such as read-only memory (ROM), flash memory, etc.), or some combination of the two. This most basic configuration is illustrated in FIG. 22 by dashed line 2206.

Computing device 2200 may have additional features/functionality. For example, computing device 2200 may include additional storage (removable and/or non-removable) including, but not limited to, magnetic or optical disks or tape. Such additional storage is illustrated in FIG. 22 by removable storage 2208 and non-removable storage 2210.

Computing device 2200 typically includes a variety of computer readable media. Computer readable media can be any available media that can be accessed by the device 2200 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 2204, removable storage 2208, and non-removable storage 2210 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by computing device 2200. Any such computer storage media may be part of computing device 2200.

Computing device 2200 may contain communication connection(s) 2212 that allow the device to communicate with other devices. Computing device 2200 may also have input device(s) 2214 such as a keyboard, mouse, pen, voice input device, touch input device, etc. Output device(s) 2216 such as a display, speakers, printer, etc. may also be included. All these devices are well known in the art and need not be discussed at length here. The functions of the various functional elements, logical blocks, modules, and circuits elements described in connection with the implementations disclosed herein may be implemented in the general context of computer executable instructions, such as software, control modules, logic, and/or logic modules executed by the processing unit. Generally, software, control modules, logic, and/or logic modules include any software element arranged to perform particular operations. Software, control modules, logic, and/or logic modules can include routines, programs, objects, components, data structures and the like that perform particular tasks or implement particular abstract data types. An implementation of the software, control modules, logic, and/or logic modules and techniques may be stored on and/or transmitted across some form of computer-readable media. In this regard, computer-readable media can be any available medium or media useable to store information and accessible by a computing device(s). Some implementations also may be practiced in distributed computing environments where operations are performed by one or more remote processing device(s)s that are linked through a communications network. In a distributed computing environment, software, control modules, logic and/or logic modules may be located in both local and remote computer storage media including memory storage device(s).

Accordingly, in an implementation, the present disclosure provides a computer implemented method for targeted patient interactions including receiving, by a processor, a patient identifier, wherein the patient identifier contains identifying data for a new patient; collecting, by at least one channel, patient data associated with the new patient, wherein the patient data is collected from at least one partner service; analyzing, by an analytical engine, the patient data; and generating, by the processor, at least one targeted patient event, wherein the targeted patient event is generated by the analysis of the patient data.

In another implementation, the computer-implemented method includes selecting patients from any one of a telecommunications, retail, patient products, pharma, financial, and healthcare context. The implementations, however, are not limited in this context.

In another implementation, the computer-implemented method of further includes managing analytics of unstructured data. The implementations, however, are not limited in this context.

In yet another implementation, the computer-implemented method further includes monetizing the unstructured data with additional context to create a social Patients persona that support the case. The implementations, however, are not limited in this context.

In still another implementation, the computer implemented method further includes a predictive analytics engine for creating or monetizing branding campaigns and demand generation. The implementations, however, are not limited in this context.

Additionally, it is to be appreciated that the implementations described herein illustrate example implementations, and that the functional elements, logical blocks, modules, and circuits elements may be implemented in various other ways which are consistent with the described implementations. Furthermore, the operations performed by such functional elements, logical blocks, modules, and circuits elements may be combined and/or separated for a given implementation and may be performed by a greater number or fewer number of components or modules. As will be apparent to those of skill in the art upon reading the present disclosure, each of the individual implementations described and illustrated herein has discrete components and features which may be readily separated from or combined with the features of any of the other several aspects without departing from the scope of the present disclosure. Any recited method can be carried out in the order of events recited or in any other order, which is logically possible.

It is noted that any reference to “an implementation” or “an implementation” means that a particular feature, structure, or characteristic described in connection with the implementation is included in at least an implementation. The appearances of the phrase “in an implementation” or “in one aspect” in the specification are not necessarily all referring to the same implementation.

Unless specifically stated otherwise, it may be appreciated that terms such as “processing,” “computing,” “calculating,” “determining,” or the like, refer to the act and/or processes of a computer or computing Exchange, or similar electronic computing device(s), such as a general purpose processor, DSP, ASIC, FPGA. PLD or other programmable logic device(s), discrete gate or transistor logic, discrete hardware components, or any combination thereof designed to perform the functions described herein that manipulates and/or transforms data represented as physical quantities (e.g., electronic) within registers and/or memories into other data similarly represented as physical quantities within the memories, registers or other such information storage, transmission or display device(s).

It is noted that some implementations may be described using the expression “coupled” and “connected” along with their derivatives. These terms are not intended as synonyms for each other. For example, some implementations may be described using the terms “connected” and/or “coupled” to indicate that two or more elements are in direct physical or virtual contact with each other. The term “coupled,” however, also may mean that two or more elements are not in direct contact with each other, but yet still co-operate or interact with each other. With respect to software elements, for example, the term “coupled” may refer to interfaces, message interfaces, application program interface (API), exchanging messages, and so forth.

It will be appreciated that those skilled in the art will be able to devise various arrangements that although not explicitly described or shown herein, embody the principles of the present disclosure and are included within the scope thereof. Furthermore, all examples and conditional language recited herein are principally intended to aid the reader in understanding the principles described in the present disclosure and the concepts contributed to furthering the art and are to be construed as being without limitation to such specifically recited examples and conditions. Moreover, all statements herein reciting principles, aspects, and implementations as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present disclosure, therefore, is not intended to be limited to the exemplary aspects and aspects shown and described herein. Rather, the scope of present disclosure is embodied by the appended claims.

The terms “.a” and “an” and “the” and similar referents used in the context of the present disclosure (especially in the context of the following claims) is to be construed to cover both the singular and the plural unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as when it was individually recited herein. All methods described herein can be performed in any suitable order unless otherwise indicated herein or otherwise clearly contradicted by context. The use of any and all examples, or exemplary language (e.g., “such as,” “in the case,” “by way of example”) provided herein is intended merely to better illuminate the disclosed implementations and does not pose a limitation on the scope otherwise claimed. No language in the specification should be construed as indicating any non-claimed element essential to the practice of the claimed subject matter. It is further noted that the claims may be drafted to exclude any optional element. As such, this statement is intended to serve as antecedent basis for use of such exclusive terminology as solely, only and the like in connection with the recitation of claim elements, or use of a negative limitation.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

While certain features of the implementations have been illustrated as described above, many modifications, substitutions, changes and equivalents will now occur to those skilled in the art. It is therefore to be understood that the appended claims are intended to cover all such modifications and changes as fall within the scope of the disclosed implementations.

What is claimed:

1. A computer implemented method for targeted patient interactions including:
receiving, by a processor, a patient identifier, wherein the patient identifier contains identifying data for a new patient;
collecting, by at least one channel, patient data associated with the new patient, wherein the patient data is collected from at least one partner service;
analyzing, by an analytical engine, the patient data; and
generating, by the processor, at least one targeted patient event, wherein the targeted patient event is generated by the analysis of the patient data.

2. The computer implemented method of claim 1, further including selecting patients from any one of a telecommunications, retail, patient products, pharma, financial, and healthcare context.

3. The computer implemented method of claim 1, further including managing analytics of unstructured data.

4. The computer implemented method of claim 3, further including a predictive analytics engine for creating or monetizing branding campaigns and demand generation for Health Care interaction and quality.

5. The computer implemented method of claim 1, further comprising:
aggregating the patent data collected from the at least one channel into a single view of the patient; and
determining, in real-time, patent needs for health services from the single view.

6. The computer implemented method of claim 1, further comprising:
determining at least one analytical indicator to derive normative discourse measures;
determining how the new patient is reacting to the targeted patient event; and
adjusting a patient interaction over the at least one channel in accordance with the patient event.

7. The computer implemented method of claim 1, further comprising:
developing a patient data model of a patient persona that is coupled to patient interactions; and
providing targeted services in accordance with the patient data model.

8. The computer implemented method of claim 1, wherein the at least one channel includes wearables, IoT devices, electronic health records, customer relation management (CRM) systems, social media, and prescription systems.

9. The computer implemented method of claim 1, further comprising applying predictive analytics provide a visualization of the patient data.

10. The computer implemented method of claim 1, further comprising:
providing a single source for the patient data; and
providing contextual view of the patient data in a user interface.

11. A health exchange platform system, comprising:
a cloud infrastructure layer that includes storage capabilities and networking capabilities;
a data management layer that includes an Application Programming Interface (API) and communication layer, a business process management layer, a rules layer, an ID management layer, a data access layer, a Lightweight Directory Access Protocol (LDAP) layer, and a database federation layer,
wherein a patient identifier containing identifying data for a new patient is received by the cloud infrastructure layer,
wherein patient data associated with the new patient and at least one channel and at least one partner service is collected by the data management layer, and
wherein an analytical engine in the data management layer analyzes the patient data to generate at least one targeted patient event from the patient data.

12. The health exchange platform system of claim 11, further wherein patients are selected from any one of a telecommunications, retail, patient products, pharma, financial, and healthcare context.

13. The health exchange platform system of claim 11, wherein the data management layer further manages analytics of unstructured data.

14. The health exchange platform system of claim 13, wherein the data management layer further includes a predictive analytics engine for creating or monetizing branding campaigns and demand generation for Health Care interaction and quality.

15. The health exchange platform system of claim 11, wherein the data management layer further aggregates the patent data collected from the at least one channel into a single view of the patient, and
wherein patent needs for health services from the single view are determined in near real-time.

16. The health exchange platform system of claim 11, wherein the data management layer further determines at least one analytical indicator to derive normative discourse measures, and determines how the new patient is reacting to the targeted patient event, and
wherein the data management layer further adjusts a patient interaction over the at least one channel in accordance with the patient event.

17. The health exchange platform system of claim 11, wherein the data management layer further develops a patient data model of a patient persona that is coupled to patient interactions and provides targeted services in accordance with the patient data model.

18. The health exchange platform system of claim 11, wherein the at least one channel includes wearables, IoT devices, electronic health records, customer relation management (CRM) systems, social media, and prescription systems.

19. The health exchange platform system of claim 11, wherein the data management layer further applies predictive analytics provide a visualization of the patient data.

20. The health exchange platform system of claim 11, wherein a single source for the patient data is provided, and
wherein a contextual view of the patient data is presented in a user interface.
